SEQUENCE LISTING (16 sequences)

SEQ ID NO: 1 - protein, 555 aa, Homo sapiens [sequence omitted]

SEQ ID NO: 2 - protein, 562 aa, Saccharomyces cerevisiae
MTIPHMKYAVSKTSENKVSNTVSPTQDKDAIRKQPDDIINNDEPSHKKIKVAQPDSLRETNTTDPLGHTKAALGEVASMELKPTNDMDPLAVSAASVVSMSNDVLKPETPKGPIIISKNPSNGIFYGPSFTKRESLNARMFLKYYGAHKFLDTYLPEDLNSLYIYYLIKLLGFEVKDQALIGTINSIVHINSQERVQDLGSAISVTNVEDPLAKKQTVRLIKDLQRAINKVLCTRLRLSNFFTIDHFIQKLHTARKILVLTGAGVSTSLGIPDFRSSEGFYSKIKHLGLDDPQDVFNYNIFMHDPSVFYNIANMVLPPEKIYSPLHSFIKMLQMKGKLLRNYTQNIDNLESYAGISTDKLVQCHGSFATATCVTCHWNLPGERIFNKIRNLELPLCPYCYKKRREYFPEGYNNKVGVAASQGSMSERPPYILNSYGVLKPDITFFGEALPNKFHKSIREDILECDLLICIGTSLKVAPVSEIVNMVPSHVPQVLINRDPVKHAEFDLSLLGYCDDIAAMVAQKCGWTIPHKKWNDLKNKNFKCQEKDKGVYVVTSDEHPKTL

SEQ ID NO: 3 - protein, 257 aa, Mus musculus
MVGAGISTPSGIPDFRSPGSGLYSNLQQYDIPYPEAIFELGFFFHNPKPFFMLAKELYPGHYRPNVTHYFLRLLHDKELLLRLYTQNIDGLERASGIPASKLVEAHGTFVTATCTVCRRSFPGEDIWADVMADRVPRCAVCTGVVKPDIVFFGEQLPARFLLHMADFALADLLLILGTSLEVEPFASLSEAVQKSVPRLLINRDLVGPFVLSPRRKDVVQLGDVVHGVERLVDLLGWTQELLDLMQRERGKLDGQDR

SEQ ID NO: 4 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 5 - protein, 399 aa, Homo sapiens; identical to SEQ ID NO: 4

SEQ ID NO: 6 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 7 - protein, 314 aa, Homo sapiens; identical to SEQ ID NO: 6

SEQ ID NO: 8 - protein, 369 aa, Homo sapiens [sequence omitted]

SEQ ID NO: 9 - protein, 389 aa, Mus musculus
MAEPDPSDPLETQAGKVQEAQDSDSDTEGGATGGEAEMDFLRNLFTQTLGLGSQKERLLDELTLEGVTRYMQSERCRKVICLVGAGISTSAGIPDFRSPSTGLYANLEKYHLPYPEAIFEISYFKKHPEPFFALAKELYPGQFKPTICHYFIRLLKEKGLLLRCYTQNIDTLERVAGLEPQDLVEAHGTFYTSHCVNTSCRKEYTMGWMKEKIFSEATPRCEQCQSVVKPDIVFFGENLPSRFFSCMQSDFSKVDLLIIMGTSLQVQPFASLISKAPLATPRLLINKEKTGQTDPFLGMMMGLGGGMDFDSKKAYRDVAWLGDCDQGCLALADLLGWKKELEDLVRREHANIDAQSGSQAPNPSTTISPGKSPPPAKEAARTKEKEEQQ

SEQ ID NO: 10 - protein, 352 aa, Rattus norvegicus
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 11 - protein, 299 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFSHLISISSLIIIKN

SEQ ID NO: 12 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 13 - protein, 357 aa, Artificial Sequence
  Synthetically generated peptide
MSVNYAAGLSPYADKGKCGLPEIFDPPEELECKVWELARLMWQSSTVVFHTGAGISTASGIPDFRGPHGVWTMEERGLAPKFDITFENARPSKTHMALVQLERMGFLSFLVSQNVDGLHVRSGFPRDKLAELHGNMFVEECPKCKTQYVRDTVVGTMGLKATGRLCTVAKARGLRACRGELRDTILDWEDALPDRDLTLADEASRTADLSVTLGTSLQIRPSGNLPLATKRRGGRLVIVNLQPTKHVCASALPSAPVSPACPLRRTDCPSLWQDRQADLCIHGYVDEVMCKLMKHLGLEIPTWDGPRVLEKALPPLPRPVAPKAEPPVHLNGSYKPKPDSPVPHRPPKRVKTEAAAS

SEQ ID NO: 14 - protein, 416 aa, Artificial Sequence
  Synthetically generated peptide
MAAGGGLSRSERKAAERVRRLREEQQRERLRQVSRILRKAAAERSAEEGRLLAESEDLVTELQGRSRRREGLKRRQEEASRGQRVCDDPEELRRKVRELAGAVRSARHLVVYTGAGISTAASIPDYRGPNGVWTLLQKGRPVSAADLSEAEPTLTHMSITQLHKHKLGLPRTAISELHGNMYIEVSSAQRTQGLGDKQMSLTVPSLPQVCTSCIPNREYVRVFDVTERTALHRHLTGRTCHKCGTQLRDTIVHFGERGTLGQPLNWEAATEAASKADTILCLGSSLKVLKKYPRLWCMTKPPSRRPKLYIVNLQWTPKDDWAALKLHGKCDDVMRLLMDELGLEIPVYNRWQDPIFSLATPLRAGEEGSHSRKSLCRSREEPPPGDQSAPLASATPILGGWFGRGCAKRAKRKKAA

SEQ ID NO: 15 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 16 - protein, 19 aa, Artificial Sequence
  Synthetically generated peptide
  VARIANT 15: Xaa = acetilated Lys
HLKSKKGQSTSRHKXLMFK

(19) United States
(12) Patent Application Publication
(10) Pub. No.: US 2006/0292652 A1
(43) Pub. Date: Dec. 28, 2006

(54) Substrate detection assay

(75) Inventors: Rory A. Curtis, Ashcand, MA (US); Andrew Napper, Salem, MA (US); Jeffrey Hixon, Salisbury, MA (US); Thomas McDonagh, Acton, MA (US)

Correspondence Address: FISH & RICHARDSON PC, P.O. BOX 1022, MINNEAPOLIS, MN, 55440-1022, US

(73) Assignee: ELIXIR PHARMACEUTICALS, INC.

(21) Appl. No.: 11/181,453

(22) Filed: Jul. 13, 2005

Related U.S. Application Data
(63) Continuation-in-part of application No. PCT/US04/01239, filed on Jan. 16, 2004.
(60) Provisional application No. 60/440,723, filed on Jan. 16, 2003.
(60) Provisional application No. 60/588,900, filed on Jul. 16, 2004.
(60) Provisional application No. 60/668,212, filed on Apr. 4, 2005.

(51) Int. Cl. C12Q 1/48; C12Q 1/26

(52) U.S. Cl. 435/15; 435/25

(57) ABSTRACT

Methods and compositions for evaluating nicotinamide-releasing activities as well as cell and organism-based evaluation methods are provided herein.

### CROSS-REFERENCE TO RELATED APPLICATIONS

This application is a continuation-in-part of PCT US2004/001239, filed on Jan. 16, 2004, and which claims the benefit of priority of U.S. Ser. No. 60/440,723, filed Jan. 16, 2003; this application also claims the benefit of priority under 35 U.S.C. § 119 of U.S. Ser. Nos. 60/588,900, filed on Jul. 16, 2004 and 60/668,212, filed Apr. 4, 2005. The contents of all of the foregoing applications are hereby incorporated by reference in their entirety.

### BACKGROUND

The pyridine nucleotides NAD⁺ and NADH are found in all living cells and serve as critical activating factors for numerous enzymatic processes. NAD⁺ consists of an adenosine monophosphate (AMP) and a nicotinamide ring. The nicotinamide ring can accept two electrons and a proton to form NADH. This reduced form readily transfers a hydride ion due to reduced resonance stability.

NADH and NAD⁺ play a central role in oxidative catabolism. They also have important non-redox activities as cellular effectors and metabolic regulators. Central to the many diverse biological activities of NAD⁺ is the enzyme-catalyzed cleavage of the nicotinamide-ribosyl bond of NAD⁺ and the attendant transfer of the ADP-ribosyl moiety to acceptors and release of nicotinamide. NAD-dependent enzymes include deacetylases, DNA ligases, aldehyde dehydrogenases, and toxins associated with cholera, diphtheria, pertussis. Other NAD-dependent activities include the reversible ADP-ribosylation-mediated biological regulatory systems; the synthesis of poly(ADP-ribose) in response to DNA damage or cellular division; and the synthesis of cyclic ADP-ribose as part of an independent, calcium-mediated regulatory system (Oppenheimer, Mol Cell Biochem 1994 September; 138(1-2):245-51).

### SUMMARY

In one aspect, the disclosure features a method of evaluating a sample. The method includes: providing a sample (e.g., including a known or unknown activity), and a donor substrate that includes (i) a nicotinamide moiety (e.g., NAD), (b) maintaining the sample under preselected conditions; (c) contacting the sample to a matrix that preferentially interacts with the donor substrate relative to nicotinamide; and (d) evaluating (i′) components of the contacted sample that do not interact with the matrix or (ii′) components of the contacted sample that do interact with the matrix.

In one embodiment, the sample further includes: (ii) a nicotinamide-releasing activity; and optionally (iii) a test compound. The method can be used to evaluate one or more test compounds.

The evaluating can include determining a parameter characteristic of components of the contacted sample that do not interact with the matrix. For example, the parameter is a function of nicotinamide concentration (e.g., directly proportional to). The method can further include comparing the parameter to a reference value, e.g., a corresponding value for a control sample evaluated by the method. A control sample can be a sample lacking the activity, a sample having a known activity, a sample lacking a donor substrate, a sample which is not subjected to the contacting step of the method, a sample which is not contacted to the matrix, etc. The control sample is otherwise identical to the sample evaluated by the method and/or is subjected to a method which is otherwise identical.

In one embodiment, the contacting includes separating components of the contact sample that interact with the matrix from the matrix, thereby separating the donor substrate from the sample.

In one embodiment, the matrix covalently bonds to the donor substrate.

In one embodiment, the donor substrate is labelled. The detecting can include detecting the label. A label can be judiciously located, e.g., depending on whether molecules that interact with the matrix or molecules that do not interact with the matrix are evaluated. For example, the nicotinamide moiety portion of the donor substrate is labeled, or non-nicotinamide moiety portion is labeled. In one embodiment, the label is radioactive. In another embodiment, the label is not radioactive. For example, the label is fluorescent.

In one embodiment, the evaluating includes an enzyme-based assay. The assay can include a deamidase such as nicotinamide deamidase or a transferase such as nicotinamide N-methyl transferase assay.

In one embodiment, the evaluating includes spectroscopic detection. In one embodiment, the separating does not require high-pressure liquid chromatography. For example, the separating can include one or more of: vacuum, gravity, or centrifugation. The method can include using a container that includes a plurality of samples, e.g., at least 6, 10, 32, 64, or 100, or 300 samples.

In one embodiment, the matrix selectively interacts with (e.g., binds or couples to) compounds having 1,2-diols. For example, the matrix can include a boronate group attached thereto. The boronate group can be attached to the resin through a variety of linker moieties X as shown below.

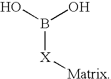

X can include an aromatic moiety such as the aniline moiety below.

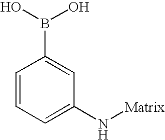

Additionally, X can include an alkyl moiety, an alkenyl moiety, an alkynyl moiety, a heteroaryl moiety, a cyclyl moiety, a heterocycly moiety, etc. In instances where the linker moiety is attached to the matrix through an amide bond, the linker moiety generally includes a nitrogen containing moiety (e.g., an amine containing moiety such as an analine) or a carbonyl moiety. In instance where the linker moiety is attached to the matrix through an ester bond, the linker moiety generally includes an oxygen containing moiety (e.g., a hydroxy containing moiety such as a phenol) or a carbonyl moiety. While amide and ester attachments are described herein, other means of attachment of the linker moiety to the matrix are also envisioned.

In one embodiment, the donor substrate is NAD, NADH, NADP, or NADPH, derivatives thereof, or a cofactor that can bind to a SIRT protein or an ADP ribosylase, or other nicotinamide modifying enzyme.

In one embodiment, the sample further includes an acetylated polypeptide, e.g., an acetylated peptide with fewer than 32, 20, or 15 amino acids. In another example, the acetylated polypeptide includes a SIRT polypeptide substrate. For example, the acetylated polypeptide includes 3, 4, 5, 11, 20, 40, or more amino acids (e.g., all amino acids) from a transcription factor (e.g., p53, a FOXO factor), a histone (e.g., H3, H4, H2A, or H2B), a cytochrome, a cytoskeletal protein, a mitochondrial protein, a protein that mediates apoptosis, that regulates cell proliferation, or that regulates senescence.

The acetylated polypeptide can also have fewer than 20, 18, 15, 12, or 10 amino acids. Exemplary peptide substrates include peptides that have at one or more lysine (K) residues, e.g., K370, K371, K372, K381, and/or K382 of human p53, or lysine positions in a non-human p53. In one embodiment, the peptide is residues 379-382 of p53 (Arg-His-Lys-Lys(Ac)). Another exemplary peptide substrate includes the acetylated N-terminal tail of a histone, e.g., H3 or H4. For example, the substrate can be the following tail of histone H4 (12-16, Lys-Gly-Gly-Ala-Lys(Ac))

Exemplary peptides include between 3 and 20, 3 and 12, 4 and 12, or 5 and 8 amino acids. Exemplary peptides can include exactly one lysine that is acetylated and/or exactly one lysine total.

The nicotinamide-releasing activity can be associated with a deacetylase activity (e.g. ability to catalyze deacetylation of an acetylated polypeptide, e.g., an acetylated lysine of a polypeptide), e.g., a deacetylase activity is associated with a histone deacetylase activity, a SIRT activity, or an activity that can deacetylate p53, a histone (e.g., H3, H4, H2A, or H2B), a cytochrome, a cytoskeletal protein, a mitochondrial protein, a protein that mediates apoptosis, that regulates cell proliferation, or that regulates senescence.

The releasing activity can be associated with a nicotinamide releasing enzyme such as CD38, CD157, poly[ADP-ribose] polymerase (PARP), or an NAD glycohydrolase, or an enzymatically active fragment of any of the aforegoing. The enzyme can also be a SIRT polypeptide or a sirtuin.

In one embodiment, the nicotinamide-releasing activity is a NAD hydrolase activity.

In one embodiment, a plurality of samples is provided and wherein a plurality of test compounds are evaluated by the method. The compounds can be compounds from a library, e.g., a library of test compounds described herein.

In one embodiment, the reaction mixture further includes an acceptor substrate, wherein the nicotinamide-releasing activity causes the acceptor substrate to become ADP-ribosylated. For example, the acceptor substrate is acetylated.

In one embodiment the test compound is stilbene or a derivative thereof, chalcone or a derivative thereof, or flavone a derivative thereof. Examples of stilbene derivatives include trans-stilbene, and hydroxy containing-trans-stilbene derivatives such as hydroxy-trans-stilbene, dihydroxy-trans-stilbene, trihydroxy-trans-stilbene (e.g., 3,5,4′-trihydroxy-trans-stilbene), tetrahydroxy-trans-stilbene (e.g., 3,5,3′,4′-tetrahydroxy-trans-stilbene), etc. Examples of chalcone derivatives hydroxy containing chalcone derivatives such as hydroxychalcone, dihydroxychalcone, trihydroxychalcone (e.g., 4,2′,4′-trihydroxychalcone), tetrahydroxychalcone (3,4,2′4′-tetrahydroxychalcone), etc. Examples of flavone derivatives include hydroxy containing flavones such as hydroxyflavone, dihydroxyflavone, trihydroxyflavone, tetrahydroxyflavone (3,7,3′,4′-tetrahydroxyflavone), pentahydroxyflavone (3,5,7,3′,4′-pentahydroxyflavone) etc. While hydroxy containing derivatives of stilbene, chalcone and flavone have been described, other substituents are also envisioned, including but not limited to halo, alkyl, alkenyl, and alkoxy.

The method can further include formulating the test compound as a pharmaceutical. The method can further include administering the test compound or a formulation thereof to a subject, e.g., a mammalian subject, e.g., a mouse, or a human, e.g., a diseased subject, an adult subject, or another subject described herein. The method can further include contacting the test compound to a cell, e.g., a mammalian cell.

In one embodiment, the nicotinamide releasing activity can include a purified protein, e.g., a recombinant protein. For example, the protein can include CD38, CD157, poly[ADP-ribose] polymerase (PARP), or an NAD glycohydrolase, or an enzymatically active fragment of any of the aforegoing. In one embodiment, the recombinant protein is tagged.

In one embodiment, the purified protein is a mutant protein (e.g., a protein that includes a polypeptide having an amino acid with one or more insertions, deletions, or substitutions relative to another sequence, e.g., a naturally-occurring sequence or other reference sequence). A mutant protein can be made by mutagenesis of a template nucleic acid that encodes a reference protein. The method can include evaluating one or more mutant proteins using a method described herein. The mutant protein can include a fragment, e.g., at least 20, 40, 50, or 80 amino acids of another protein, e.g., a functional fragment.

In one embodiment, the sample is a patient sample or a fraction thereof (e.g., a membrane fraction of a cellular extract, a protein-clarified extract, a nuclear extract, or a cytoplasmic extract). For example, a sample from a polypeptide can be contacted with an antibody that is specific for a polypeptide that has nicotinamide releasing activity, e.g., a SIRT protein, to provide a fraction enriched (or at least 10% purified for the SIRT protein).

In one embodiment, the nicotinamide releasing activity is immobilized during the maintaining, e.g., immobilized to a planar substrate, a bead, a reaction vessel, etc.

Exemplary preselected conditions include a preselected temperature or temperature range, e.g., between 0-50, 4-42, 10-40, 10-20, 20-30, 25-40, 30-40, 30-35, 35-40, or 36-39° C. Exemplary conditions can include a preselected pH, e.g., between 5-9, 6-8, 5-7, or 7-9.

In a related aspect, the disclosure features a method of evaluating a sample. The method includes: providing a sample (e.g., including a known or unknown activity), e.g., the sample including (i) a compound that includes a ribose containing moiety, (b) maintaining the sample, e.g., under preselected conditions; (c) contacting the sample to a matrix that preferentially interacts with a reactant relative to a reaction product, or that preferentially interacts with a reaction product relative to a reactant; and (d) evaluating components of the contacted sample that do not interact with the matrix, or components of the sample that do interact with the matrix.

In the case of a matrix that can bind to other ribose containing moieties, the matrix can be used to evaluate a reaction (e.g., an enzymatic reaction) in which the compound that includes a ribose containing moiety is modified. For example, the method can be used evaluate a reaction (or activity of a reaction component, e.g., an enzyme) for ability to modify the compound. The modification may cause a moiety (e.g., a labeled moiety) to be covalently linked to the compound, or may cause a moiety (e.g., a labeled moiety) to be released from the compound (e.g., by breaking a covalent bond).

The compound may include a ribose moiety bound to a nitrogen containing heteroaryl moiety. The nitrogen containing heteroaryl moiety can be substituted, for example with an amide moiety, a hydroxy moiety, a cyano moiety, an ester moiety, a nitro moiety, etc. In some instances, the nitrogen containing heteroaryl moiety is substituted with multiple substituents. In some instances the nitrogen contining heteroaryl moiety is labeled, for example using a radioisotope or a fluorescent probe.

Examples of nitrogen containing heteroaryl moieties include, but are not limited to pyridine, pyrimidine, pyrazine, pyridazine, pyrrole, pyrrolopyrimidine, etc.

The method can include other features, e.g., other features described herein.

In another aspect, the disclosure features a method that includes: (a) providing a sample; (b) contacting the sample with a NAD-interacting matrix, wherein the matrix does not interact with nicotinamide, under conditions that allow the NAD to bind (e.g. couple to) the matrix; and (c) detecting nicotinamide after the contacting. The method can include other features described herein.

In one embodiment, the sample is obtained by lysing a cell, or combining components, e.g., purified components (one or more of the components can be at least 10, 20, 50, 70, 80, 90, 95, or 99% pure). Exemplary samples include extracts, e.g., nuclear extracts, cytoplasmic extracts, clarified extracts, lipid free extracts, protein only fraction, protein size fractionated fractions, chromatographic separation fractions, and so forth. Exemplary purified components include SIRT1 polypeptides, nicotinamide releasing enzymes such as CD38, CD157, poly[ADP-ribose] polymerase (PARP), or an NAD glycohydrolase, or an enzymatically active fragment of any of the aforegoing, and their substrates.

In another aspect, the disclosure features a method that includes: (a) providing a sample; (b) contacting the sample to a nicotinamide modifying activity; and (c) detecting a product produced by a reaction catalyzed by the nicotinamide modifying activity. The sample can include, for example, (i) a sample having nicotinamide-releasing activity; (ii) a donor substrate, wherein the donor substrate includes a nicotinamide moiety. In one embodiment, the sample further includes (iii) a test compound. Prior to the contacting, the method can further include: maintaining the sample under preselected conditions. In one embodiment, the detecting includes detecting a fluorescence or calorimetric product, e.g., using spectroscopy.

In one embodiment, the nicotinamide-modifying activity is nicotinamide deamidase activity. In one embodiment, the product is ammonia, and the ammonia is detected, e.g., by contacting the reaction mixture with o-phthaldialdehyde (OPA) (e.g., and also sodium sulfite, and sodium borate); and evaluating an optical property of the reaction mixture, e.g., fluorescence, thereby detecting levels of ammonia released.

In one embodiment, a plurality of samples is provided and a plurality of test compounds are evaluated.

In one embodiment, the nicotinamide-modifying enzyme is nicotinamide N-methyl transferase, and wherein the modified nicotinamide is detected. In another embodiment, a nicotinamide modifying activity can include a nicotinamide releasing enzymes such as CD38, CD157, poly[ADP-ribose] polymerase (PARP), or an NAD glycohydrolase, or an enzymatically active fragment of any of the aforegoing.

In one embodiment, the contacting step further includes contacting the sample with acetophenone/KOH and formic acid, and, optionally, heating the sample.

In one embodiment, the detecting includes detecting fluorescence (OD).

The method can further include determining a parameter characteristic of the detected sample. For example, the parameter is a function of nicotinamide concentration (e.g., directly proportional to nicotinamide concentration). The method can further include comparing or correlating the parameter to a reference value, e.g., a corresponding value for a control sample evaluated by the method. Exemplary control samples include a sample that is not contacted to the nicotinamide-modifying activity, a sample contacted to a known level of nicotinamide-modifying activity, a sample lacking a donor substrate, a sample lacking a test compound, etc. The control sample can be otherwise identical to the sample evaluated by the method and/or is subjected to a method which is otherwise identical.

The method can further include other features described herein.

In one aspect, the disclosure features a method of evaluating a nicotinamide-releasing activity. The method includes: (a) providing a sample including (i) nicotinamide-releasing activity, and (ii) a donor substrate, wherein the donor substrate includes a nicotinamide moiety; (b) contacting the reaction mixture with nicotinamide-modifying activity under conditions that allow the enzyme to react with nicotinamide; and (c) evaluating one or more of: a nicotinamide that has been modified by the enzyme, or a product of the nicotinamide-modifying activity. The method can include other features described herein. A related method can be used to evaluate a nicotinamide binding activity, a reaction that produces nicotinamide as a product, or a reaction that uses nicotinamide as a substrate.

In still another aspect, the disclosure features a method of detecting a deactylase activity, e.g., a histone deacetylase activity. The method includes: providing a sample having deacetylase activity (e.g., a histone deacteylase) or a sirtuin protein; maintaining the sample under preselected conditions; and detecting or evaluating nicotinamide. The sample can include an acetylated substrate of the histone deacteylase or the sirtuin protein. In one embodiment, the sample further includes a donor substrate, wherein the donor substrate includes a nicotinamide moiety.

In one embodiment, the method can further include, prior to the detecting, separating the nicotinamide from the donor substrate by binding the sample to a matrix that selectively interacts with the donor substrate but not the nicotinamide, wherein the binding is performed under conditions that allow the unreacted donor substrate to bind the matrix. In another embodiment, the method further includes contacting the sample with a nicotinamide-modifying enzyme under conditions that allow the nicotinamide-modifying enzyme to react with the nicotinamide; and detecting a product of a reaction catalyzed by the nicotinamide modifying enzyme. The method can include other features described herein.

In another aspect, the disclosure features a method of purifying a nicotinamide releasing activity. The method includes assaying one or more fraction from a separation procedure using a method described herein. The method can be used to purify a nicotinamide releasing activity, e.g., a sirtuin protein.

In another aspect, the disclosure features a kit including: a control enzyme having nicotinamide-releasing activity; a nicotinamide-modifying enzyme; and NAD.

The kit can further include: instructions for detecting one of: modified nicotinamide, or a byproduct of an activity of the nicotinamide-modifying enzyme, e.g., using a method described herein. In another aspect, the disclosure features a kit including: a nicotinamide-binding matrix; NAD, an acetylated acceptor peptide; and optionally a control enzyme and instructions for use in detecting nicotinamide-releasing activity. For example, the nicotinamide-modifying enzyme can be CD38, CD157, poly[ADP-ribose] polymerase (PARP), or an NAD glycohydrolase, or an enzymatically active fragment of any of the aforegoing. The enzyme can also be a SIRT polypeptide or a sirtuin.

In one aspect, this disclosure provides methods to evaluate the activity of nicotinamide-releasing enzymes, such as the SIRT class of deacetylases and the NAD hydrolases. SIRT enzymes are implicated in aging and age-related diseases such as cancer. The methods enable, for example, careful kinetic analysis and high-throughput testing of compounds as potential inhibitors and activators.

In one embodiment, the assay is implemented for a plurality of samples (which may include different components or different conditions, or which may include a common set of components or conditions, and one variable element (e.g., a test compound). The method can be implemented using a single plate and/or homogeneous fluorescent detection that is highly sensitive and miniaturizable. In one embodiment, the method does not use radioactive materials, thereby avoiding associated handling and disposal issues.

The disclosure also features boronate resins in a multi-sample container, e.g., a multi-well plate such as a microplate, (e.g., a Multiscreen plate from Millipore Corp). Exemplary boronate resins are commercially available from Pierce (www.piercenet.com) and Bio-Rad (www.biorad.com).

In one embodiment, a method described herein is performed without the use of column separation, e.g., without chromatography and/or without solvent extraction. The method can include elution of bound radiolabeled product before scintillation counting.

The disclosure also features a method for evaluating an enzyme that includes contacting a reaction mixture (or fraction thereof) to a boronate resin to separate nicotinamide from NAD as the basis for an enzyme assay. In another aspect, the disclosure features a method for evaluating an enzyme that includes providing nicotinamide-modifying activity to the reaction mixture (or a fraction thereof) and evaluating a product of the nicotinamide-modifying activity. In another aspect, the disclosure features direct fluorometric detection of ammonia in an enzyme assay, e.g., of an enzyme that produces a product which can be used as a substrate for ammonia detection.

In some implementations the assays of nicotinamide release allow miniaturization, e.g., to 96- and 384-well microplate format, protein array, protein chip, or micro-chip formats. In many implementations, the methods are non-radioactive, homogeneous methods. The fluorescent readouts are highly sensitive and amenable to miniaturization and avoid the need to handle radioactive materials.

The nicotinamide detection method can also be used to avoid evaluating depletion of substrate, e.g., NAD depletion.

A streamlined assay for nicotinamide releasing enzymes (e.g., SIRT enzymes) can be used, for example, for enzymatic characterization and for profiling of test compounds, e.g., inhibitors, e.g., for potency and selectivity. The assays can be implemented for high-throughput screening.

A sample can be a biological sample, e.g., from an organism (e.g., blood, biopsy, cells) or from cultured cells, or a protein sample (e.g., purified protein), or a cellular extract.

In one aspect, this disclosure features a method of evaluating a compound by evaluating the effect of the compound on a parameter of a first cell that has a first level of expression or activity of a sirtuin (e.g., Sirt1, Sirt2, Sirt3, Sirt4, Sirt5, Sirt6, or Sirt7), and evaluating the effect of the compound on a parameter of a second cell that has a second level of expression or activity of the sirtuin. The method can further include identifying the compound as a candidate compound if the compound has a differential effect on the second cell compared to the first cell.

The first level of sirtuin expression or activity can be a wild-type level or any reference level. In one embodiment, the second level of sirtuin expression or activity is less than the first level, e.g., 75%, 60%, 50%, 25%, 10%, or 5% less than that of the first level of sirtuin expression or activity. In one embodiment, the second level of sirtuin expression or activity corresponds to no expression or no activity. In another embodiment, the second level of sirtuin expression or activity is greater than that of the first level of sirtuin expression or activity.

A variety of strategies can be used to obtain the first and second cell. The first and second cell can be both be related to a common parental cell, e.g., a normal or so-called wild-type cell. One of the first and second cell can be modified to alter sirtuin expression, e.g., to increase or decrease sirtuin expression. For example, the second cell contains RNA that interferes with expression of the sirtuin, e.g., an siRNA and/or a DNA that produces such RNA. In another example, the first and second are obtained from different animals. For example, the first cell is derived from an animal with a wild-type sirtuin, and the second cell is derived from an animal with a mutant sirtuin.

The cell is typically a eukaryotic cell, e.g., a yeast cell or an animal cell, e.g., a mammalian cell, e.g., a mouse or human cell. The cell can be maintained in culture, e.g., as a immortalized cell or a primary cell. In some embodiments, the cell is a skin cell, pancreatic cell, adipose cell, nerve cell, or a cell from some other tissue. In one embodiment, the cell is a fibroblast cell, e.g., a embryonic fibroblast cell.

The effect of a test compound on the first and second cell is compared by evaluating a parameter in both cells. The evaluations can be performed sequentially or concurrently. The parameter can be based on assessment of a cellular function, e.g., proliferation, metabolism, intracellular signalling, inter-cellular signalling, and apoptosis. In one embodiment, the parameter is based on assessment of proliferation, e.g., the parameter is a rate of proliferation, which can be assessed, e.g., by assessing incorporation of radiolabeled thymidine. In another embodiment, the parameter relates to secretion of signalling protein, e.g., a hormone or growth factor such as insulin. In one embodiment, the parameter is uptake of a molecule, e.g., a nutrient such as glucose. In one embodiment, the parameter evaluated is the acetylation state of a protein, e.g., a nuclear protein, e.g., a histone or transcription factor, e.g., Tat, p53, or a Forkhead transcription factor, e.g., Foxo1, Foxo3. In one embodiment, the parameter is an assessment of transcriptional expression of a target gene in a sirtuin pathway, e.g., using a reporter construct operably linked to a sirtuin-regulated promoter.

In one embodiment, the method further includes, e.g., before, during, or after, evaluating one of the cells, contacting the compound to a sirtuin in vitro, and evaluating an interaction between the compound and the sirtuin. In one embodiment, the method further includes assaying the candidate compound for effects on Sirt1 enzymatic activity, e.g., in vitro.

The method can be used to evaluate members of a library of compounds. A subset of members (e.g., one or more members) can be selected based on the evaluation. For example, compounds that show at threshold differential effect, e.g., a statistically significant differential effect can selected.

Another aspect of this disclosure features a method of evaluating a compound by evaluating the effect of the compound on a parameter of a first cell that has a first level of expression or activity of a protein; evaluating the effect of the compound on a parameter of a second cell that has a second level of expression or activity of the protein; and identifying the compound as a candidate compound if the compound has a differential effect on the second cell compared to the first cell. The protein can be one that modulates lifespan regulation, e.g., Na⁺-coupled citrate transporter (NaCT), AMP-activated protein kinase (AMP-K), and insulin-like growth factor receptor (IGF-1R), a component of the AMPK pathway, or a component of the GH pathway.

In one embodiment, the first level of expression or activity of the protein is greater than the second level of expression or activity of the protein. In one embodiment, the first level of expression or activity of the protein is a wild-type level. In one embodiment, the second level of expression or activity of the protein is less than 75%, 60%, 50%, 25%, 10%, or 5% of that of the first level of expression or activity of the protein. In one embodiment, the second level of expression or activity of the protein corresponds to no expression or no activity. In one embodiment, the second level of expression or activity of the protein is greater than the first level of expression or activity of the protein.

The cell is typically a eukaryotic cell, e.g., a yeast cell or an animal cell, e.g., a mammalian cell, e.g., a mouse or human cell. The cell can be maintained in culture, e.g., as a immortalized cell or a primary cell. In some embodiments, the cell is a skin cell, pancreatic cell, adipose cell, nerve cell, or a cell from some other tissue. In one embodiment, the cell is a fibroblast cell, e.g., a embryonic fibroblast cell.

In one embodiment, the first cell is derived from an animal with a wild-type protein, and the second cell is derived from an animal with a mutant protein. In one embodiment, the second cell contains RNA that interferes with expression of the protein, e.g., an siRNA.

In one embodiment, the parameter evaluated is proliferation rate, e.g., by incorporation of radiolabeled thymidine. In one embodiment, the parameter evaluated is secretion of, e.g., insulin. In one embodiment, the parameter evaluated is apoptosis. In one embodiment, the parameter evaluated is uptake of a molecule, e.g., citrate. In one embodiment, the parameter measured is transcriptional activation or repression, e.g., using a reporter construct. In one embodiment, the parameter measured is cell motility.

In one embodiment, the method further includes, before or after evaluating one of the cells, contacting the compound to Na⁺-coupled citrate transporter (NaCT), AMP-activated protein kinase (AMP-K), or insulin-like growth factor receptor (IGF-1R) in vitro, and evaluating an interaction between the compound and the sirtuin. In one embodiment, the method further includes assaying the candidate compound for effects on activity of Na⁺-coupled citrate transporter (NaCT), AMP-activated protein kinase (AMP-K), and insulin-like growth factor receptor (IGF-1R). In one embodiment, activity of Na⁺-coupled citrate transporter (NaCT), AMP-activated protein kinase (AMP-K), or insulin-like growth factor receptor (IGF-1R) is measured in vitro. In one embodiment, the parameters of the first and second cells are evaluated in parallel.

The first and second cells can be provided together, e.g., in a kit that includes a container for each of the cells. The kit can further include instructions for using the cells, e.g., in screening assays.

In another aspect, the disclosure features a method for screening to evaluate a test compound, e.g., to identify activators and repressors of sirtuins., e.g., in a cell free system.

In a first embodiment, sirtuin protein activity is evaluated in an assay that includes evaluating a sample (e.g., a substrate in a sample) using mass spectroscopy, e.g., to produce a mass spectroscopy readout. For example, a sirtuin protein, a test compound, and optionally one or more cofactors are combined to provide a sample and maintained under conditions that allow sirtuin enzymatic activity. The mixture can also include an acetylated substrate (e.g., an acetylated lysine amino acid, an acetylated histone or transcription factor, e.g., p53, or a fragment of such proteins). Mass spectroscopy can be used to evaluate conversion of acetylated protein into non-acetylated protein. Other physical techniques can also be used to evaluate the acetylated proteins.

In a second embodiment, sirtuin protein activity is evaluated in an assay that includes detecting proteolytic fragments following deacetylation of a substrate. For example, the assay can include maintaining a mixture under conditions which allow sirtuin activity, and then trypsinisation (e.g. non-deacetylated protein will not be cleaved at this site, as in fluor-de-lys assay, Biomol). Trypsinization of the deacetylated product can produce a signal relative to trypsinization of the corresponding acetylated product.

In another embodiment, a reaction by-product is evaluated. For example, it is possible to evaluate the sample for O-acetyl-ADP-ribose released during deacetylation reaction or to evaluate the sample for ADP-ribose.

Cell-based screens can be used to evaluate a test compound, e.g., to identify activators and repressors of sirtuins.

In one embodiment, the test compound is contacted to a microorganism cell, e.g., a yeast cell, e.g., in yeast cell-based screen. The contacted cell can be evaluated for a parameter, e.g., silencing of URA3, 5FOA conversion, survival readout, or other transcriptional readouts.

In one embodiment, the test compound is contacted to a metazoan cell, e.g., a mammalian cell, e.g., a mouse cell. For example, it is possible to evaluate wild-type mouse cells (e.g. stressor/survival assay) for a phenotypic property, using SIRT1 KO mouse cells as control or counter screen. In addition to evaluate the cells, extracts from such cells can be evaluated, e.g., using mass Spectroscopy (MS/MS) or other method to detect deacetylation of proteins in cell extracts. In another example, the cell includes a reporter gene which indicates deacetylation activity in the cell, e.g., a reporter gene for a gene regulated by a sirtuin, e.g., Sirt1.

Still other methods for evaluating test compounds include methods for screening, e.g., ones particularly suited for inhibitor screening. For example, it is possible to use Fleur-de-Lys™ to evaluate test compounds as inhibitors of certain sirtuins. Enzymes can be obtained from commercially available sources or expressed and purified. An exemplary substrate is the p53 Fleur-de-Lys™ substrate which can be used with other reagents (proteolytic developer), e.g., those commercially available.

Generally, for any sirtuin substrate, one can identify an acetylated lysine peptide substrate and modify the peptide with quencher and fluorphore. Deacetylation of lysine and trypsinisation alters activity of the fluorophore.

The terms “modulated” and “differentially regulated” include increasing (including, for example, activation or stimulation) and decreasing (including, for example, inhibition or suppression) relative to a reference level.

The GH pathway is defined and described, e.g., in Ser. No. 10/656,530. The AMPK pathway is defined and described, e.g., in PCT/US03/38628.

“Nicotinamide-releasing activity” refers to an activity that produces nicotinamide (or a molecule that includes nicotinamide-containing moiety) from a precursor compound (e.g., NAD). Generally the activity is an enzymatic activity. For example, the nicotinamide-releasing activity can be an enzyme, e.g., an enzyme that can interact with NAD, NADP, NADH, a ribonucleoside, a ribonucleotides, or nicotinamide. “Nicotinamide-modifying activity” refers to an activity that causes modification of nicotinamide molecules or of molecules that include a nicotinamide moiety.

The term “matrix” refers to any insoluble support, e.g., a particle (e.g., a magnetic particle, porous particle), a bead, a planar surface, a resin, a gel (e.g., agarose, or polyacrylamide gel), all or part of a reaction vessel such as a multi-container sample carrier (e.g., a microtitre plate), tube, column, spin-cup, disposable pipet tip, ring, disc (e.g., paper disc), membrane. For example, the templates can be attached to a surface within one or more microtitre wells (e.g., in a variety of formats, including single, strips, 96-well, 384-well, robotically manipulated single or multiple plates).

The term “polypeptide” refers to a polymer of three or more amino acids linked by a peptide bond. The polypeptide may include one or more unnatural amino acids. Typically, the polypeptide includes only natural amino acids. The term “peptide” refers to a polypeptide that is between three and thirty-two amino acids in length. A “protein” can include one or more polypeptide chains. Accordingly, the term “protein” encompasses polypeptides and peptides. A protein or polypeptide can also include one or more modifications, e.g., an acetylation, glycosylation, amidation, phosphorylation, and so forth.

The term “isolated nucleic acid molecule” or “purified nucleic acid molecule” includes nucleic acid molecules that are separated from other nucleic acid molecules present in the natural source of the nucleic acid. For example, with regards to genomic DNA, the term “isolated” includes nucleic acid molecules which are separated from the chromosome with which the genomic DNA is naturally associated. In some embodiments, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Examples of flanking sequences include adjacent genes, transposons, and regulatory sequences. Moreover, an “isolated” nucleic acid molecule, such as a cDNA molecule, can be substantially free of other cellular material, of culture medium when produced by recombinant techniques, or of chemical precursors or other chemicals when chemically synthesized.

As used herein, a “naturally-occurring” nucleic acid molecule refers to an RNA or DNA molecule having a nucleotide sequence that occurs in Nature. For example a naturally occurring nucleic acid molecule can encode a natural protein.

As used herein, the terms “gene” and “recombinant gene” refer to nucleic acid molecules which include at least an open reading frame encoding a protein, a protein subunit, derivative, or functional domain thereof. The gene can optionally further include non-coding sequences, e.g., regulatory sequences and introns.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that the protein of interest in the preparation is at least 10% pure. In an embodiment, the preparation of the protein has less than about 30%, 20%, 10% and more preferably 5% (by dry weight), of a contaminating component (e.g., a protein not of interest, chemical precursors, and so forth). When the protein or biologically active portion thereof is recombinantly produced, it is also preferably substantially free of culture medium, i.e., culture medium represents less than about 20%, more preferably less than about 10%, and most preferably less than about 5% of the volume of the protein preparation. Also featured are isolated or purified preparations of at least 0.01, 0.1, 1.0, and 10 milligrams in dry weight.

Fragments can be evaluated for enzymatic activity using any standard assay including those described herein. Fragments that retain at least 5% of the enzymatic activity of the full length mature protein are considered enzymatically active.

In one embodiment, the methods described herein can be used to evaluate “SIRT proteins” and “SIRT polypeptides” are used interchangeably herein and refer to members of the Silent Information Regulator (SIR) family of genes. In particular, the term “SIRT1 protein” or “SIRT1 polypeptide” refers to a polypeptide that is at least 25% identical to a conserved SIRT catalytic domain, amino acid residues 258 to 451 of SEQ ID NO:1 (shown in Table 1, below), and likewise for other SIRT proteins known or described herein (see, e.g., Table 2).

TABLE 1
Human SIRT1 Amino Acid Sequence

GenBank ® GI: 9884660, Acc:CAC04174.1|bA57G10.4
(SIRT1, Sir2-like proteins (siruitins) type 1)
(Homo sapiens)

(SEQ ID NO:1)

MIGTDPRTILKDLLPETIPPPELDDMTLWQIVINILSEPPKRKKRKDINT
IEDAVKLLQECKKIIVLTGAGVSVSCGIPDFRSRDGIYARLAVDFPDLPD
PQAMFDIEYFRKDPRPFFKFAKEIYPGQFQPSLCHKFIALSDKEGKLLRN
YTQNIDTLEQVAGIQRIIQCHGSFATASCLICKYKVDCEAVRGDIFNQVV
PRCPRCPADEPLAIMKPEIVFFGENLPEQFHRAMKYDKDEVDLLIVIGSS
LKVRPVALIPSSIPHEVPQILINREPLPHLHFDVELLGDCDVIINELCHR
LGGEYAKLCCNPVKLSEITEKPPRTQKELAYLSELPPTPLHVSEDSSSPE
RTSPPDSSVIVTLLDQAAKSNDDLDVSESKGCMEEKPQEVQTSRNVESIA
EQMENPDLKNVGSSTGEKNERTSVAGTVRKCWPNRVAKEQISRRLDGNQY
LFLPPNRYIFHGAEVYSDSEDDVLSSSSCGSNSDSGTCQSPSLEEPMEDE
SEIEEFYNGLEDEPDVPERAGGAGFGTDGDDQEAINEAISVKQEVTDMNY
PSNKS

Other exemplary SIRT proteins include SIRT2 and SIRT3.

In some embodiments, a SIRT polypeptide can be at least 30, 40, 50, 60, 70, 80, 85, 90, 95, 99% homologous to a reference sequence. In other embodiments, the SIRT1 polypeptide can be a fragment, e.g., a fragment of SIRT1 capable of one or more of: deacetylating a substrate in the presence of NAD and/or a NAD analog and capable of binding a target protein, e.g., a transcription factor, e.g., p53 or a transcription factor other than p53. Such functions can be evaluated, e.g., by the methods described herein. In other embodiments, the SIRT polypeptide can be a “full length” SIRT polypeptide. The term “full length” as used herein refers to a polypeptide that has at least the length of a naturally-occurring SIRT polypeptide (or other protein described herein). A “full length” SIRT polypeptide or a fragment thereof can also include other sequences, e.g., a purification tag, or other attached compounds, e.g., an attached fluorophore, or cofactor. The term “SIRT polypeptides” can also include sequences or variants that include one or more substitutions, e.g., between one and ten substitutions, with respect to a naturally occurring Sir2 family member. In preferred embodiments, a human SIRT polypeptide can vary from SEQ ID NO:1 by at least 1, 2, 3, 4, 5, 10, 15, but preferably not more than 20 to 50 amino acid residues, e.g., it can vary by at least 1, 2, 3, 4, 5, 10, 15 substitutions, e.g., conservative substitutions. In other embodiments, the SIRT1 polypeptide is encoded by a nucleic acid which hybridizes under stringent conditions to a nucleic acid encoding the amino acid sequence of SEQ ID NO:1, e.g., at least amino acid residues 258 to 451 of SEQ ID NO:1. The term “SIRT polypeptide” is also includes homologs of human SIRT proteins from other species including the murine homolog of SIRT1, also referred to as “Sir2α”. A “SIRT1 activity” refers to one or more activity of SIRT1, e.g., deacetylation of transcription factors such as p53 or histone proteins, (e.g., in the presence of a cofactor such as NAD and/or an NAD analog) and binding of a target protein, e.g., a transcription factor, e.g., p53 or a transcription factor other than p53.

A variety of methods can be used to identify a SIRT family member. For example, a known amino acid sequence of a known SIRT protein can be searched against the GenBank® sequence databases (National Center for Biotechnology Information, National Institutes of Health, Bethesda Md.), e.g., using BLAST; against Pfam database of HMMs (Hidden Markov Models) (using default parameters for Pfam searching; against the SMART database; or against the ProDom database. For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314. The SMART database (Simple Modular Architecture Research Tool, EMBL, Heidelberg, DE) of HMMs as described in Schultz et al. (1998), Proc. Natl. Acad. Sci. USA 95:5857 and Schultz et al. (200) Nucl. Acids Res 28:231. The SMART database contains domains identified by profiling with the hidden Markov models of the HMMer2 search program (R. Durbin et al. (1998) Biological sequence analysis: probabilistic models of proteins and nucleic acids. Cambridge University Press). The database also is annotated and monitored. The ProDom protein domain database consists of an automatic compilation of homologous domains. (Corpet et al. (1999), Nucl. Acids Res. 27:263-267) Current versions of ProDom are built using recursive PSI-BLAST searches (Altschul et al. (1997) Nucleic Acids Res. 25:3389-3402; Gouzy et al. (1999) Computers and Chemistry 23:333-340.) of the SWISS-PROT 38 and TREMBL protein databases. The database automatically generates a consensus sequence for each domain.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. The comparison is generally done using a Blossum 62 scoring matrix with a gap penalty of 12, a gap extend penalty of 4, and a frameshift gap penalty of 5.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to reference nucleic acid molecules. BLAST protein searches can be performed with the XBLAST program, score=50, wordlength=3 to obtain amino acid sequences homologous to reference protein molecules. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Some polypeptides can have an amino acid sequence substantially identical to an amino acid sequence described herein. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. Methods described herein can include use of a polypeptide that includes an amino acid sequence that contains a structural domain having at least about 60%, or 65% identity, likely 75% identity, more likely 85%, 90%, 92%, 94%, 95%, 96%, 97%, 98% or 99% identity to a domain of a polypeptide described herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. Methods described herein can include use of a nucleic acid that includes a region at least about 60%, or 65% identity, likely 75% identity, more likely 85%, 90%. 91%, 92%, 93%, 94%, 95%, 96%, 97%, 98% or 99% identity to a nucleic acid sequence described herein, or use of a protein encoded by such nucleic acid.

A “non-essential” amino acid residue is a residue that can be altered from the wild-type sequence of protein without abolishing or substantially altering activity, e.g., the activity is at least 20%, 40%, 60%, 70% or 80% of wild-type. An “essential” amino acid residue is a residue that, when altered from the wild-type sequence results in abolishing activity such that less than 20% of the wild-type activity is present. Conserved amino acid residues are frequently predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another embodiment, mutations can be introduced randomly along all or part of a coding sequence, such as by saturation mutagenesis, and the resultant mutants can be screened for biological activity to identify mutants that retain activity. Following mutagenesis, the encoded protein can be expressed recombinantly and the activity of the protein can be determined.

As used herein, a “biologically active portion” or a “functional domain” of a protein includes a fragment of a protein of interest which participates in an interaction, e.g., an intramolecular or an inter-molecular interaction, e.g., a binding or catalytic interaction. An inter-molecular interaction can be a specific binding interaction or an enzymatic interaction (e.g., the interaction can be transient and a covalent bond is formed or broken). An inter-molecular interaction can be between the protein and another protein, between the protein and another compound, or between a first molecule and a second molecule of the protein (e.g., a dimerization interaction). Biologically active portions/functional domains of a protein include peptides comprising amino acid sequences sufficiently homologous to or derived from the amino acid sequence of the protein which include fewer amino acids than the full length, natural protein, and exhibit at least one activity of the natural protein. Biological active portions/functional domains can be identified by a variety of techniques including truncation analysis, site-directed mutagenesis, and proteolysis. Mutants or proteolytic fragments can be assayed for activity by an appropriate biochemical or biological (e.g., genetic) assay. In some embodiments, a functional domain is independently folded.

Exemplary biologically active portions of an enxymatic protein can include at least a minimal enzymatic core domain that has an active site and detectable enzymatic activity in vitro. Exemplary biologically active portions include between 5-100% of the reference protein, e.g., between 10-99, 10-95, 15-94, 15-90, 20-90, 25-80, 25-70, 25-60, 25-50, 25-40, 5-25, or 75-90% of the reference protein. Biologically active portions can include, e.g., internal deletions, insertions (e.g., of a heterologous sequence), terminal deletions, and substitutions. Typically, biologically active portions comprise a domain or motif with at least one activity of the protein, e.g., a nicotinamide-releasing activity or an NAD-dependent activity.

“NAD” refers to nicotinamide adenine dinucleotide. An “NAD analog” as used herein refers to a compound (e.g., a synthetic or naturally occurring organic molecule) which possesses structural similarity to component groups of NAD (e.g., adenine, ribose and phosphate groups) or functional similarity. For example, an NAD analog can be 3-aminobenzamide or 1,3-dihydroisoquinoline (H. Vaziri et al., EMBO J. 16:6018-6033 (1997)).

The details of one or more embodiments of the inventions are set forth in the accompanying drawings and the description below. Other features, objects, and advantages of the inventions will be apparent from the description and drawings, and from the claims. All cited patents, patent applications (published and unpublished), and references (including references to public sequence database entries) are incorporated by reference in their entireties for all purposes. U.S. Ser. No. 60/440,723, filed Jan. 16, 2003, U.S. Ser. No. 10/191,121 (published as US20040005574-A1), and U.S. Ser. No. 09/461,580 (published as US20030207325-A1) are incorporated by reference in their entireties for all purposes.

### DESCRIPTION OF DRAWINGS

FIG. 1 is a schematic depiction of the chemical structures of ¹⁴C-NAD, ¹⁴C-NAD bound to a boronate-based affinity resin, and ¹⁴C-nicotinamide in steps of an exemplary nicotinamide release assay in which nicotinamide is quantitated after flowing through the affinity resin.

FIG. 2 is a schematic diagram of an exemplary nicotinamide release assay in which ¹⁴C-NAD and an acetylated peptide are incubated with a sample such that ¹⁴C-nicotinamide, peptide, and O-actetyl-ADP-ribose are generated by an activity in the sample. The supernatant of the sample is diluted with ammonium acetate and transferred to a filter plate in which it is mixed with a boronate resin. The supernatant is then filtered, and ¹⁴C-nicotinamide in the filtrate is quantitated.

FIG. 3 is a schematic depiction of the chemical structures of ¹⁴C-NAD, ¹⁴C-NAD bound to a boronate-based affinity resin, and ¹⁴C-nicotinamide in steps of an exemplary nicotinamide release assay in which nicotinamide is quantitated after separation from unreacted ¹⁴C-NAD which is bound to the affinity resin.

FIG. 4 is a schematic diagram of an exemplary nicotinamide release assay in which ¹⁴C-NAD and an acetylated peptide are incubated with a sample such that ¹⁴C-nicotinamide, peptide, and O-actetyl-ADP-ribose are generated by an activity in the sample. The sample is mixed with a boronate resin and diluted with ammonium acetate. After the resin settles, the supernatant is removed and nicotinamide in the removed supernatant is quantitated.

FIG. 5 is a schematic depiction of the chemical structures of NAD, nicotinamide, and nicotinic acid in steps of an exemplary nicotinamide release assay. The ammonia generated by the activity of nicotinamide deamidase on nicotinamide is detected with a fluorometric assay after reaction with o-phthaldealdehyde (OPA), sodium sulfite, and sodium borate.

FIG. 6 is a schematic diagram of an exemplary nicotinamide release assay in which NAD and an acetylated peptide are incubated with a sample such that nicotinamide, peptide, and O-acetyl-ADP-ribose are generated. Next, the sample is incubated with nicotinamide deamidase. OPA is added and incubated with the sample, and fluorescence is detected.

FIG. 7 is a schematic depiction of the chemical structures of NAD, nicotinamide, N-methyl nicotinamide, and the product of N-methyl nicotinamide after reaction with acetophenone/KOH and formic acid.

FIG. 8 is a schematic diagram of an exemplary nicotinamide release assay in which NAD and acetylated peptide are incubated with a sample such that nicotinamide, peptide, and O-acetyl-ADP-ribose are generated. Next, the sample is incubated with nicotinamide N-methyl transferase (NNMT). Acetophenone/KOH and formic acid are added, and fluorescence in the reaction mixture is detected.

FIG. 9 is a graph depicting the results of an exemplary nicotinamide release assay in which samples containing ¹⁴C-NAD or ¹⁴C-nicotinamide were mixed with a boronate resin. The samples were filtered, and counts per minute (CPM) in the filtrate and total mixture were detected.

FIG. 10A and FIG. 10B are graphs depicting the results of an exemplary nicotinamide release assay in which ¹⁴C-NAD and an acetylated peptide, p53-382, were incubated with human SIRT1. Cpm of ¹⁴C-nicotinamide was determined after the reacted sample was filtered through a boronate resin. In FIG. 10A, the results are depicted as cpm versus the concentration of the substrate peptide. In FIG. 10B, the results are depicted as cpm versus the concentration of NAD.

FIG. 11 is a schematic depicting an example of the screening method.

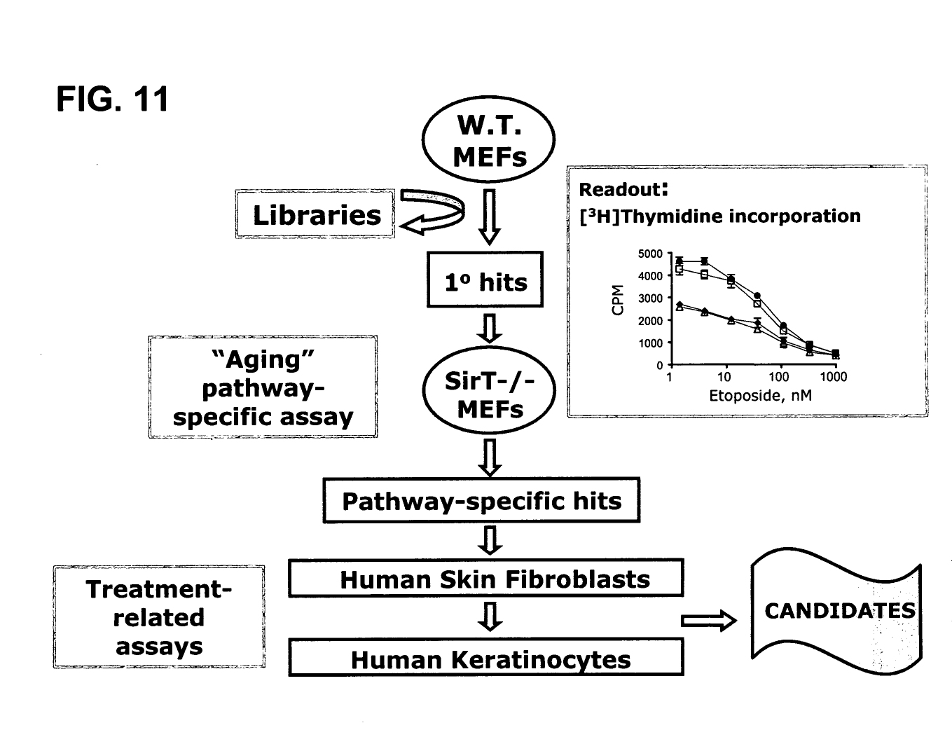

FIG. 12 is a schematic depicting an example of the screening method for Sirt1.

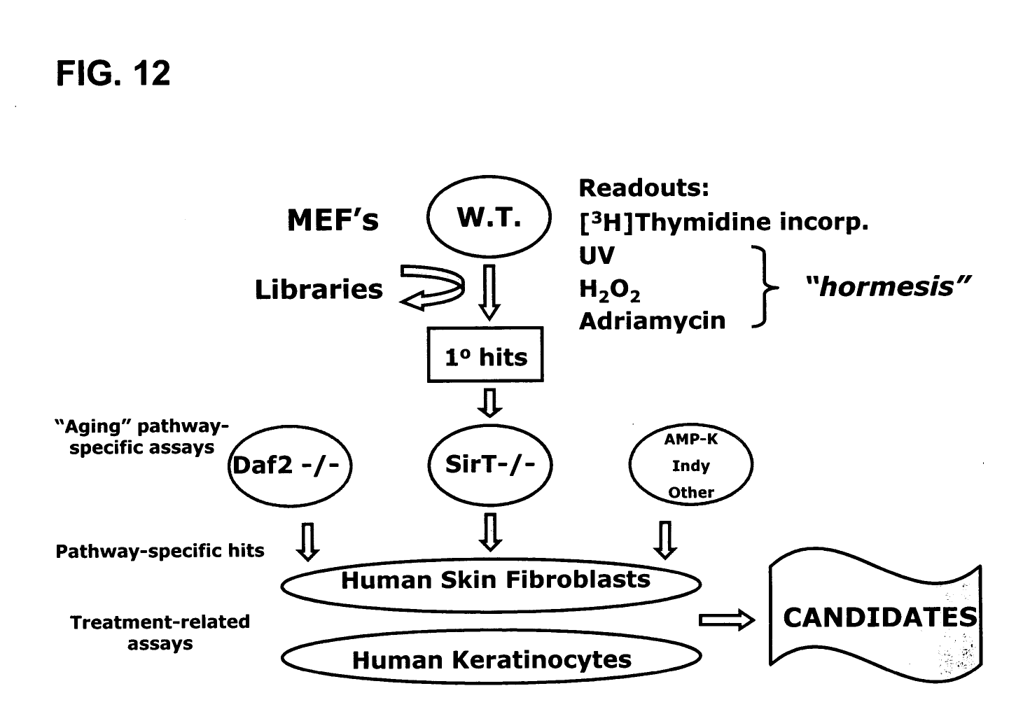

### DETAILED DESCRIPTION

In one aspect, this disclosure provides evaluation techniques that can be used to evaluate nicotinamide and other related compounds. In another aspect, the disclosure provides a method of screening compounds, e.g., using cells that have different levels of expression or activity of the pathway or target protein. The methods can also be using in conjuction with the methods for evaluating nicotinamide and other related compounds.

With resepect, to the methods for evaluating nicotinamide and other related compounds, in some implementations, the assays can be used to detect release of nicotinamide, e.g., by an enzyme. The assays are useful for evaluating enzymes directly or indirectly, e.g., by detecting the release of nicotinamide. For example, the activity of NAD-dependent enzymes can be evaluated with these assays. Exemplary NAD-dependent enzymes include NAD-hydrolases, deacetylases, DNA ligases, aldehyde dehydrogenases, and toxins, e.g., toxins associated with cholera, diphtheria, pertussis.

The assays can also be used to study other enzymes that cleave NAD to nicotinamide. These include CD38, a type I transmembrane protein (M. Howard, J. C. Grimaldi, J. F. Bazan, F. E. Lund, L. Santos-Argumedo, R. M. Parkhouse, T. F. Walseth, H. C. Lee, Science 262 (1993) 1056-9; K. Kontani, H. Nishina, Y. Ohoka, K. Takahashi, T. Katada, J. Biol. Chem. 268 (1993) 16895-8); CD157/bone marrow stromal antigen (BST1), a GPI-linked extrinsic membrane protein (C. Dong, J. Wang, P. Neame, M. D. Cooper, Int. Immunol. 6 (1994) 1353-60); nuclear poly [ADP-ribose] polymerase (PARP) enzymes (P. Chambon, Weil, J. D., Mandel, P., Biochem. Biophys. Res. Comm. 11 (1963) 39-43; D. D'Amours, S. Desnoyers, I. D'Silva, G. G. Poirier, Biochem. J. 342 (1999) 249-68); and streptococcal NAD glycohydrolase, a secreted protein which is a potential virulence factor for the pathogenic bacterium Streptococcus pyogenes responsible for toxic shock syndrome and necrotizing fasciitis in humans (D. L. Stevens, D. B. Salmi, E. R. McIndoo, A. E. Bryant, J. Infect. Dis. 182 (2000) 1117-28), or enzymatically active fragments thereof.

The assays described here include, for example, assays in which a sample is contacted to a matrix that selectively binds a precursor of nicotinamide (e.g., NAD), and that does not bind nicotinamide, such that nicotinamide generated in a sample (e.g., by an enzymatic reaction) can be separated from the matrix. Other exemplary assays detect nicotinamide after treatment with a nicotinamide-modifying enzyme. Enzymes such as nicotinamide deamidase and nicotinamide N-methyl transferase react with nicotinamide and produce detectable compounds, or precursors of detectable compounds.

Nicotinamide-Releasing Enzymes

Exemplary nicotinamide-releasing enzymes include NAD-dependent histone deacetylases (which may have substrates other than histones), the SIRT class of deacetylases, as well as other nicotinamide-releasing enzymes. Nicotinamide-releasing enzymes can include NAD-dependent histone deacetylases (e.g., Sir2), NAD hydrolases, NAD glycohydrolases (Balducci and Micossi, Mol Cell Biochem. 233(1-2):127-32, 2002), NAD(+) glycohydrolases (Tono-oka and Hatakeyama, Chem Pharm Bull.50(6):831-3, 2002), Poly(ADP-ribose) polymerase (Ha et al., Proc Natl Acad Sci USA 99(1):245-50, 2002), and bacterial toxins (e.g., diphtheria toxin, Kahn and Bruice, J Am Chem Soc. 123(48):11960-9, 2001). Examples of nicotinamide releasing enzymes include CD38, CD157, poly[ADP-ribose] polymerase (PARP), or an NAD glycohydrolase, or an enzymatically active fragment of any of the aforegoing.

For example, as described in Munshi et al, J. Biol. Chem., Vol. 275, Issue 28, 21566-21571, Jul. 14, 2000, an enzymatic fragment (or catalytic domain) of CD38 has been characterized. Catalytic residues span over 100 residues in the CD38 sequence, from Trp-125 to Glu-226. The catalytic domain is also described in pfam02267.11 as “Rib_hydrolayse.” An exemplary fragment of CD157 includes amino acids 30-148 of the human CD157 amino acid sequence.

NAD-Dependent Histone Deacetylases and Substrates

NAD-dependent histone deacetylases can catalyze a NAD-nicotinamide exchange reaction that requires the presence of acetylated lysines such as those found in the N termini of histones. These enzymes are distinguished from other classes of deacetylases in that the histone deacetylation reaction absolutely requires NAD. The enzymes are active on histone substrates that have been acetylated by both chromatin assembly-linked and transcription-related acetyltransferases. These enzymes may also ADP-ribosylate histones.

Examples of NAD-dependent histone deacetylases include Sir2 of S. cerevisiae (reviewed in Guarente, 2000; Shore, 2000) and Sir2 homologs (Imai et al., 2000; Smith et al., 2000). Deacetylation of acetyl-lysine by Sir2 is tightly coupled to NAD hydrolysis. It has been reported that this reaction produces nicotinamide and a novel acetyl-ADP ribose compound (1-O-acetyl-ADP-ribose) (Tanner et al., 2000; Landry et al., 2000b; Tanny and Moazed, 2001).

The novel assays described herein are exemplified by Assay A, Assay B, Assay C, and Assay D, described below. While the assays are depicted with SIRT as a nicotinamide-releasing enzyme, it is understood that other nicotinamide-releasing enzymes and may be evaluated in these assays.

Assay A: Filtration Assay of ¹⁴C-nicotinamide Release

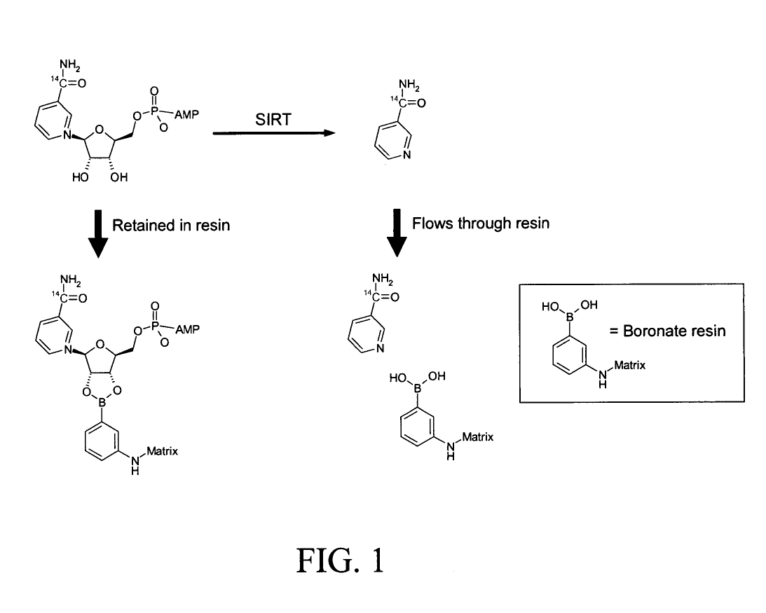

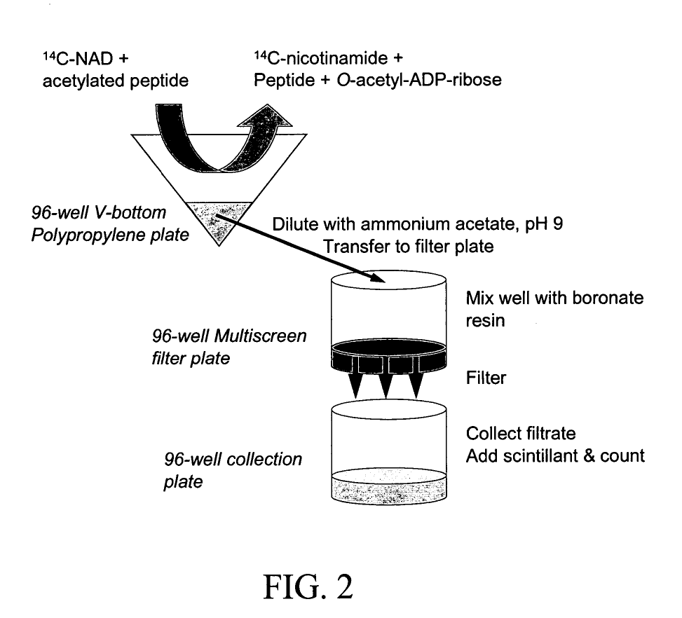

This method is based on the use of boronate-based affinity resin that selectively binds 1,2-diols. ¹⁴C-labeled NAD and acetylated substrate are incubated with SIRT enzyme. Following the enzymatic reaction, the release of ¹⁴C-nicotinamide from NAD may be quantified by filtration of the reaction mixture through boronate resin. The chemical structures of NAD and nicotinamide in the steps of the assay are depicted in FIG. 1. The resin selectively binds excess unreacted NAD while allowing nicotinamide to flow through unbound (FIG. 2).

B: Resin-Binding Assay of ¹⁴C-nicotinamide Release

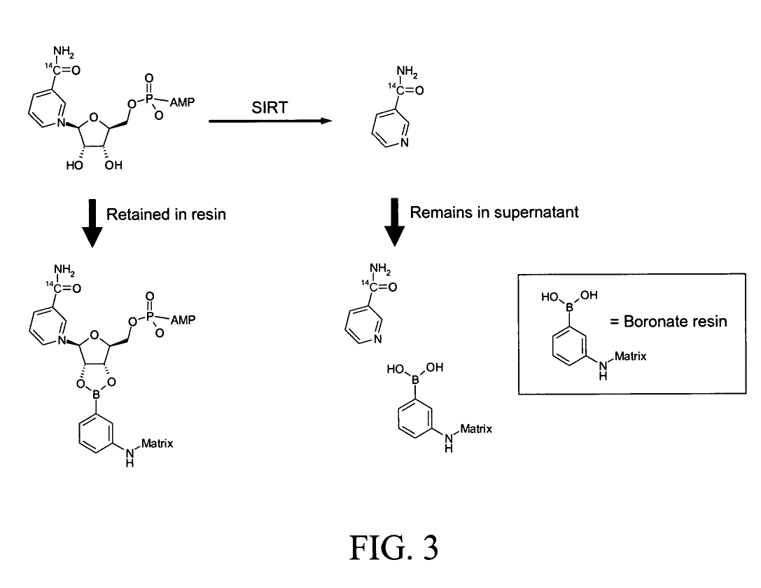

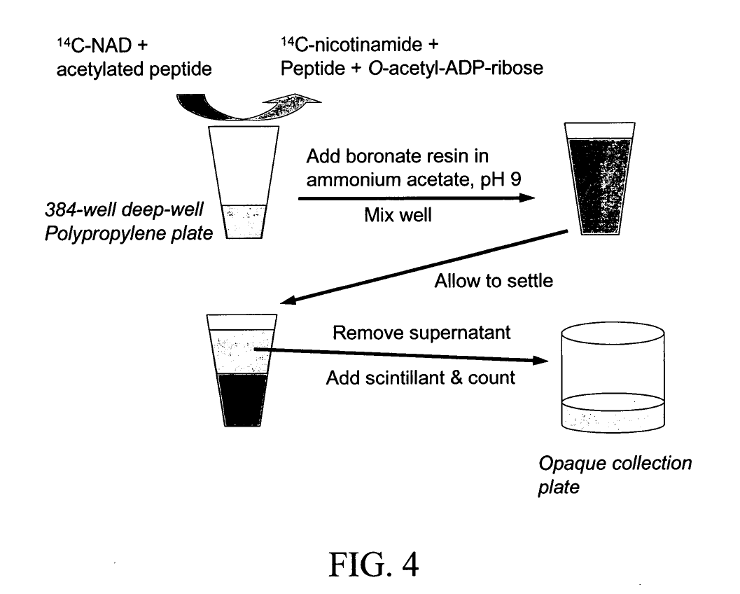

This method is the same as Assay A except that the enzymatic reaction and the resin binding are performed in the same plate. Following the enzymatic reaction, a slurry of resin is added, mixed well, and allowed to settle. The chemical structures of NAD and nicotinamide in the steps of the assay are depicted in FIG. 3. The resin selectively binds excess unreacted NAD while allowing nicotinamide to remain in the supernatant (FIG. 4). A portion of the supernatant is transferred to a second plate for quantification of ¹⁴C-nicotinamide release by scintillation counting.

C: Coupled Enzymatic Assay of Nicotinamide Release (Ammonia Detection)

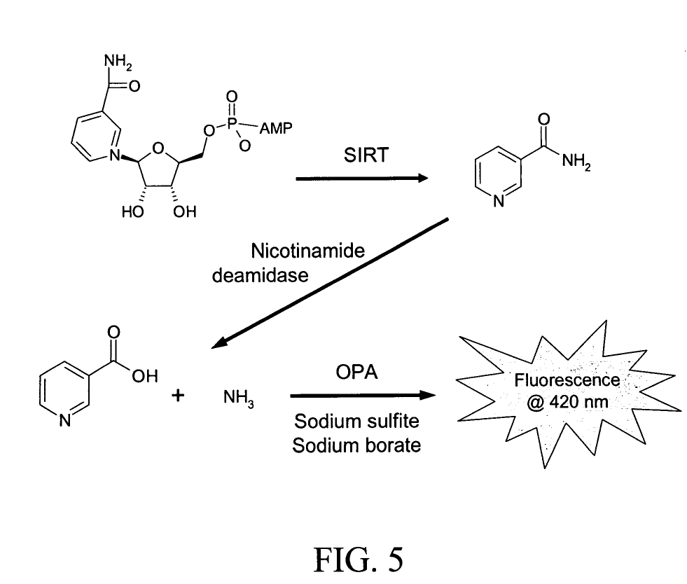

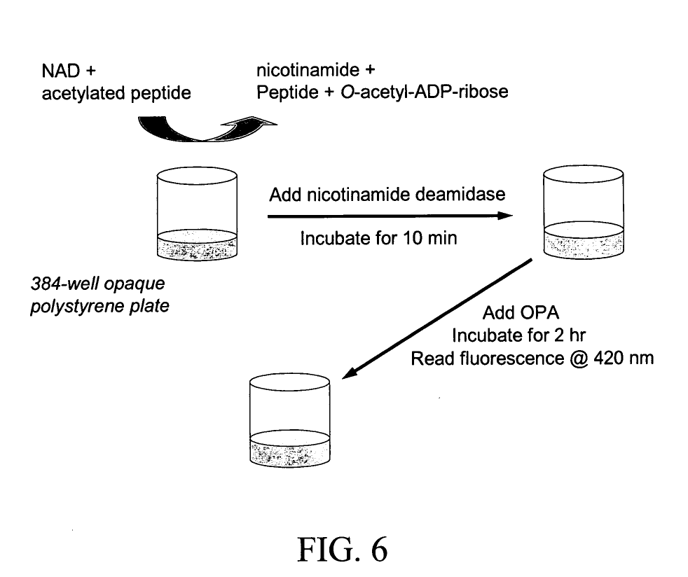

This method is based on enzymatic release of ammonia from nicotinamide and subsequent fluorometric ammonia detection. NAD and acetylated substrate are incubated with SIRT enzyme. The nicotinamide released in the SIRT reaction is then converted to nicotinic acid and ammonia by addition of the enzyme nicotinamide deamidase. The chemical structures of NAD, nicotinamide, and nicotinic acid in steps of this assay are depicted in FIG. 5. Ammonia is then detected by fluorometric assay using o-phthaldealdehyde (OPA) (FIG. 6).

D: Coupled Enzymatic Assay of Nicotinamide Release (N-methyl nicotinamide Detection)

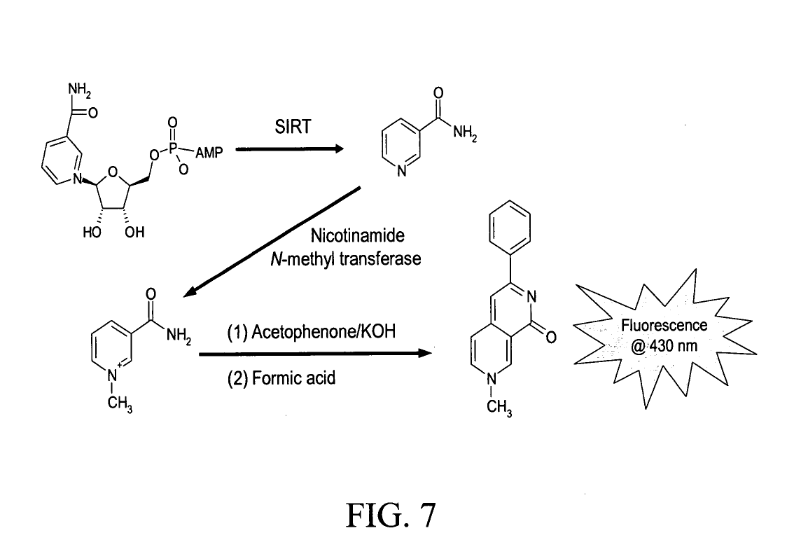

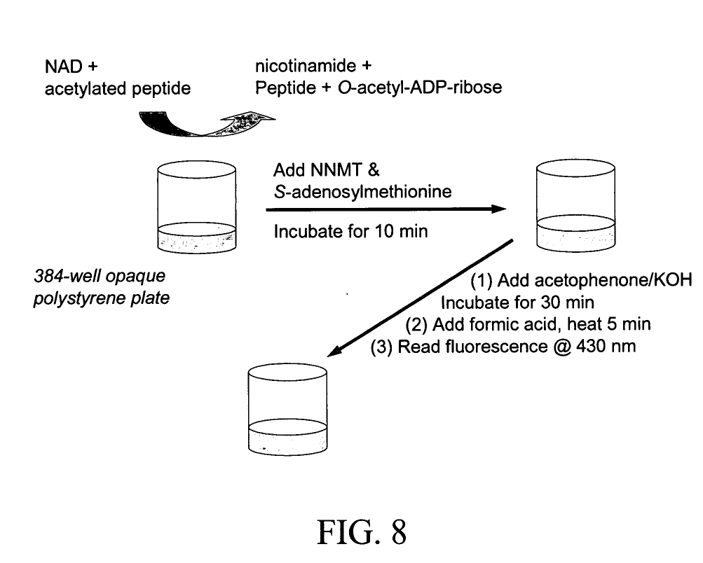

This method is based on enzymatic conversion of nicotinamide to N-methyl nicotinamide followed by reaction of the latter with acetophenone to generate a fluorescent product. The structures of NAD, nicotinamide, and products of reacted nicotinamide are shown in FIG. 7. NAD and acetylated substrate (e.g., an acetylated peptide) are incubated with SIRT enzyme. The nicotinamide released in the SIRT reaction is then converted to N-methyl nicotinamide by addition of the enzyme nicotinamide N-methyl transferase (NNMT). Base-catalyzed reaction with acetophenone followed by acidification of the condensation product with formic acid results in ring closure to form a fluorescent product (FIG. 8).

Test Compounds and Libraries

A “compound” or “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., a herb or a nature product), synthetic, or both. Examples of macromolecules are proteins, protein complexes, and glycoproteins, nucleic acids, e.g., DNA, RNA and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, organic or inorganic compounds e.g., heteroorganic or organometallic compounds. Exemplary compounds include those listed as generally recognized as safe (GRAS) by the FDA.

A test compound can be the only substance assayed by the method described herein. Alternatively, a collection of test compounds can be assayed either consecutively or concurrently by the methods described herein.

The test compounds can be obtained or produced using any of the numerous combinatorial library methods. Some exemplary libraries include: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. These approaches can be used, for example, to produce peptide, non-peptide oligomer or small molecule libraries of compounds (see, e.g., Lam (1997) Anticancer Drug Des. 12:145).

A biological library includes polymers that can be encoded by nucleic acid. Such encoded polymers include polypeptides and functional nucleic acids (such as nucleic acid aptamers (DNA, RNA), double stranded RNAs (e.g., RNAi), ribozymes, and so forth). The biological libraries and non-biological libraries can be used to generate peptide libraries. Another example of a biological library is a library of dsRNAs (e.g., siRNAs), or precursors thereof. A library of nucleic acids that can be processed or transcribed to produce double-stranded RNAs (e.g., siRNAs) is also featured.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., framework).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Test compounds can also be obtained from: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

The compound can be from a natural product or extract. The compound can be evaluated in purified form, or as a component of a more complex mixture, e.g., an extract. Exemplary natural products include:

Vitamins—A (beta-carotene or retinol), D (calciferols), E (tocopherols), K (phylloquinone), B-1 (thiamine), B-2 (riboflavin), B-6 (pyridoxine), B-12 (cobalamin), C (ascorbic acid), Biotin, Choline, folic acid (folate, B-vitamin), niacin (sometimes called vitamin B-3), pantothenic acid

Antioxidants (a variety of molecules including vitamins E, C, A and trace elements such as selenium, copper and zinc.

Minerals—calcium, iodine, iron, copper, chromium, magnesium, manganese, molybdenum, zinc, potassium, selenium, phosphorus, boron, fluorine, germanium.

Herbals/Botanicals—ginkgo biloba, Echinacea, garlic, black cohosh root, ginseng, St. John's Wort, kava kava, valerian, saw palmetto, soy, bilberry, green tea, milk thistle.

Non-Herbals—glucosamine, chondroitin, probiotics such as lactobacillus and acidophilus, DHEA (dehydroepiandosterone), CoQ-10 (Co-Enzyme Q-10), lecithin, melatonin, flax, flaxseed oil, SAMe

Other dietary supplements—proteins such as soy protein and amino acids

Non-essential amino acids—alanine, serines, L-tyrosine, glycines, L-glutamine, L-glutamic acid, L-histidine, L-cysteine, L-aspartic acid, L-ornithine, asparagine, praline, L-arginine.

Essential amino acids—threonine, L-phenylalanine, D-phenylalanine, DL-phenylalanine, L-lysine, L-leucine, L-isoleucine, L-valine, L-methionine, taurine, L-tryptophan

Functional additives—lycopene, isoflavones, tocotrienols, sterols, probiotics such as Lactobacillus acidophilus, Bifidobacterium bifidum, and Bifidobacterium longum, polyunsaturated fatty acids, fibers such as psyllium

Many natural products can be obtained from: Advanced Nutraceuticals, Inc.(US), Archer Daniels Midland Company (US), BASF AG (DE), Bayer AG (DE), Beaufour-Ipsen (FR), Ceapro, Inc. (CA), F. Hoffman La Roche AG (Switzerland), GlaxoSmithKline (UK), Laboratories Arkopharma SA (FR), Leiner Health Products (US), Mannatech, Inc. (US), Mead Johnson Nutritionals (US), Natrol, Inc. (US), NBTY, Inc. (US), Novartis AG (Switzerland), Nutraceutical International Corp. (US), Ocean Nutrition Canada (CA), Perrigo Company (US), Pharmavite Corp. (US), Rexall Sundown, Inc. (US), Royal Numico NV (Netherlands), Scolr Inc. (US), Twinlab Corp. (US), U.S. Nutraceuticals LLC (US), and Wyeth (US).

Exemplary products can be derived from a plant, fungus, bacteria, or animal, e.g., from Achillea millefolium, Arctium lappa, Arnica chamissonis, Artemisia absinthum, Astragalus membranaceus, Borago officinalis, Calendula officinalis, Catha edulis, Centaurea cyanoides, Cheiranthus cheiri, Chelidonium majus, Cichorium pumilum, Citrullus colocynthis, Cynara cardunculus, Echinacea angustifolia, Echinacea pallida, Echinacea purpurea, Eruca satvia, Eschscholzia californica, Filipendula ulmaria, Galega officinalis, Gingko biloba, Glechoma hederacea, Hypericum perforatum, Hypericum triquetrifolium, Hyssopus officinalis, Leonurus cardiaca, Lippia citriodora, Majorana syriaca, Marrubium valgare, Melissa officinalis, Mentha spicata, Mentha piperita, Mercurialis annua L., Micromeriafruticosa, Nepeta cataria, Olea europaea, Origanum vulgare, Passiflora incarnata, Plantago mayor, Rosmarinus officinalis, Ruta graveolens, Salvia hierosolymitana, Salvia officinalis, Salvia sclarea, Satureja hortensis, Satureja thymbra, Scutellaria baicalensis, Scutellaria laterifolia, Stellaria media, Stevia rabaudiana, Symphytum officinale, Tanacetum partheneum, Taraxacum officinale, Thymus hyb. lemon, Thymus vulgaris, Tribulus terrestis, Urtica urens, Valeriana officinalis, Verbascum sinuatum, Verbascum thapsus, Verbena officinalis, Vitex agnus-castus, and Withania somenifera.

In many cases, a high throughput screening approach to a library of test compounds includes one or more assays, e.g., a combination of assays. Information from each assay can be stored in a database, e.g., to identify candidate compounds that can serve as leads for optimized or improved compounds, and to identify SARs. Information from an assay described herein (e.g., a separation-based or coupled-enzyme assay) can be stored on a computer-readable medium, e.g., in a database, e.g., a relational database. The database can related parameters detected by the assay with sample information (e.g., test compound identity, reaction conditions, patient source, enzyme source (e.g., to evaluate mutated proteins for enzymatic activity), etc.).

After using an assay described herein, the test compound can be assayed in vivo or in the presence of a cell, e.g., a cultured cell. A cell-based assay can include evaluating cell proliferation, cell differentiation, and cell apoptosis.

Compounds that bind and/or modulate the activity of nicotinamide-releasing enzymes have been described. See, for example, Howitz, et. al. Nature, 425(6954): 191-6, 2003. Compounds that can be tested in the methods described herein include such compounds and derivatives thereof.

Exemplary test compounds include stilbenes and derivatives thereof, chalcones and derivatives thereof, and flavones and derivative thereof. Examples of stilbene derivatives include trans-stilbene, and hydroxy containing-trans-stilbene derivatives such as hydroxy-trans-stilbene, dihydroxy-trans-stilbene, trihydroxy-trans-stilbene (e.g., 3,5,4′-trihydroxy-trans-stilbene), tetrahydroxy-trans-stilbene (e.g., 3,5,3′,4′-tetrahydroxy-trans-stilbene), etc.

Examples of chalcone derivatives hydroxy containing chalcone derivatives such as hydroxychalcone, dihydroxychalcone, trihydroxychalcone (e.g., 4,2′,4′-trihydroxychalcone), tetrahydroxychalcone (3,4,2′4′-tetrahydroxychalcone), etc. Examples of flavone derivatives include hydroxy containing flavones such as hydroxyflavone, dihydroxyflavone, trihydroxyflavone, tetrahydroxyflavone (3,7,3′,4′-tetrahydroxyflavone), pentahydroxyflavone (3,5,7,3′,4′-pentahydroxyflavone) etc. While hydroxy containing derivatives of stilbene, chalcone and flavone have been described, other substituents are also envisioned, including but not limited to halo, alkyl, alkenyl, and alkoxy.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

Structural Activity Relationships

It is also possible to use structure-activity relationships (SAR) and structure-based design principles to find compounds that have improved effects on a target protein, e.g., a nicotinamide releasing activity, e.g., a SIRT activity. SARs provide information about the activity of related compounds in at least one relevant assay. Correlations are made between structural features of a compound of interest and an activity. For example, it may be possible by evaluating SARs for a family of compounds that interact with a target protein to identify one or more structural features required for the interaction. A library of compounds can then be produced that vary these features, and then the library is screened. Structure-based design can include determining a structural model of the physical interaction of the compound and its target. The structural model can indicate how an antagonist of the target can be engineered.

Both the SAR and the structure-based design approach can be used to identify a pharmacophore. Pharmacophores are a highly valuable and useful concept in drug discovery and drug-lead optimization. A pharmacophore is defined as a distinct three dimensional (3D) arrangement of chemical groups essential for biological activity. Since a pharmaceutically active molecule must interact with one or more molecular structures within the body of the subject in order to be effective, and the desired functional properties of the molecule are derived from these interactions, each active compound must contain a distinct arrangement of chemical groups which enable this interaction to occur. The chemical groups, commonly termed descriptor centers, can be represented by (a) an atom or group of atoms; (b) pseudo-atoms, for example a center of a ring, or the center of mass of a molecule; (c) vectors, for example atomic pairs, electron lone pair directions, or the normal to a plane. Once formulated a pharmacophore can be used to search a database of chemical compound, e.g., for those having a structure compatible with the pharmacophore. See, for example, U.S. Pat. No. 6,343,257 ; Y. C. Martin, 3D Database Searching in Drug Design, J. Med. Chem. 35, 2145(1992); and A. C. Good and J. S. Mason, Three Dimensional Structure Database Searches, Reviews in Comp. Chem. 7, 67(1996). Database search queries are based not only on chemical property information but also on precise geometric information.

Computer-based approaches can use database searching to find matching templates; Y. C. Martin, Database searching in drug design, J. Medicinal Chemistry, vol. 35, pp 2145-54 (1992), which is herein incorporated by reference. Existing methods for searching 2-D and 3-D databases of compounds are applicable. Lederle of American Cyanamid (Pearl River, N.Y.) has pioneered molecular shape-searching, 3D searching and trend-vectors of databases. Commercial vendors and other research groups also provide searching capabilities (MACSS-3D, Molecular Design Ltd. (San Leandro, Calif.); CAVEAT, Lauri, G et al., University of California (Berkeley, Calif.); CHEM-X, Chemical Design, Inc. (Mahwah, N.J.)). Software for these searches can be used to analyze databases of potential drug compounds indexed by their significant chemical and geometric structure (e.g., the Standard Drugs File (Derwent Publications Ltd., London, England), the Bielstein database (Bielstein Information, Frankfurt, Germany or Chicago), and the Chemical Registry database (CAS, Columbus, Ohio)).

Once a compound is identified that matches the pharmocophore, it can be tested for activity, e.g., for binding to a component of a target protein and/or for a biological activity, e.g., a nicotinamide-releasing activity.

Purification Methods

The assays described herein can be used to evaluate one or more fractions from a purification, e.g., a purification from a natural source (e.g., tissue samples, e.g., human, murine, or bovine tissue) or a recombinant source. Recombinant proteins can be purified from any suitable expression system. The method can be used to identify a peak of activity, e.g., a fraction that contains a nicotinamide-releasing activity, e.g., a histone deacetylase, e.g., a SIRT activity.

Proteins may be purified to substantial purity by standard techniques, including selective precipitation with such substances as ammonium sulfate; column chromatography, affinity purification, immunopurification methods, and others (see, e.g., Scopes, Protein Purification: Principles and Practice (1982); U.S. Pat. No. 4,673,641; Ausubel et al., supra; and Sambrook et al., supra). In one embodiment, recombinant Proteins can include an affinity tag that can be used for purification, e.g., in combination with other steps. For example, Crute et al. (1998) J. Biol. Chem. 273:35347-35354 describe use of a glutathione-S-transferase N-terminal tag to purify recombinant proteins.

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein. Proteins expressed in bacteria may form insoluble aggregates (“inclusion bodies”). Several protocols are suitable for purifying proteins from inclusion bodies. See, e.g., Sambrook et al., supra; Ausubel et al., supra). If the proteins are soluble or exported to the periplasm, they can be obtained from cell lysates or periplasmic preparations.

Differential Precipitation. Salting-in or out can be used to selectively precipitate a protein. An exemplary salt is ammonium sulfate. Ammonium sulfate precipitates proteins on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration precipitates many of the more hydrophobic proteins. The precipitate is analyzed to determine if the protein of interest is precipitated or in the supernatant. Ammonium sulfate is added to the supernatant to a concentration known to precipitate the protein of interest. The precipitate is then solubilized in buffer and the excess salt removed if necessary, either through dialysis or diafiltration.

Column chromatography. Proteins can be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech). See, generally, Scopes, Protein Purification: Principles and Practice (1982).

In one embodiment, each fraction (or at least a plurality of fractions) is evaluated by a method described herein. The fractions can be evaluated in parallel. The method can be used to identify a peak of activity, e.g., a fraction that contains a nicotinamide-releasing activity, e.g., a histone deacetylase, e.g., a SIRT activity.

Cell/Organism-Based Screening

This disclosure also provides, inter alia, a cell or organism-based method of screening compounds. Compounds that interact with and modulate pathways or target proteins involved in lifespan regulation, aging, or metabolism have therapeutic and prophylactic uses. Such compounds can be used to prevent, treat or otherwise ameliorate cancer, diabetes, obesity, frailty, skin aging, and neurodegenerative disorders, among other disorders associated with these pathways or target proteins.

Examples of pathways involved in lifespan regulation, aging, or metabolism include the GH axis and the AMPK pathway. Examples of target proteins include: sirtuins, sodium citrate transporters (such as INDY), AMP-K, and IGF-1R.

To determine if a test compound modulates a pathway or target protein, its effect on cells (or organism that include such cells) that have different levels of expression or activity of the pathway or target protein can be compared. Exemplary test compounds include those described above. Test compounds that have a differential effect on the cells can be chosen as lead compounds for further testing or as candidate compounds, e.g., for use in cosmetic or pharmaceutical preparations.

Cells

The cells that are used to evaluate compounds are typically mammalian cells, e.g., from a human, a mouse, rat, primate, or other non-human, but also may be non-mammalian (e.g., animal cells such as, Xenopus, zebrafish, invertebrate such as a fly or nematode; or also yeast). The cells are typically culture cells, e.g., cells that can be maintained in tissue culture, including immortalized cells or primary cells. Exemplary cell lines include those available from the American Type Culture Collection (Manassas, Va.). An exemplary cell line is the mouse fibroblast cell line 3T3. Multiple cell types can be used. For example, a compound can be evaluated using a first cell type (e.g., fibroblasts) and then using a second cell type (e.g., keratinocytes).

Primary cells can derived from animal tissues by standard methods, e.g., from mice or humans, to provide mouse or human fibroblasts. Exemplary primary fibroblast cells are mouse embryonic fibroblasts and human skin fibroblasts.

A variety of methods are available to obtain a set of cells that have different levels of expression or activity of a target protein. For example, cells can be obtained from a single source and split into separate aliquots. One aliquot is modified, e.g., to increase or decrease expression or activity of the target protein. The other aliquot can either be unmodified or can be modified with a control or other neutral treatment. For example, one of the aliquots is modified by introduction of nucleic acid, e.g., by transfection of a cell with a construct, e.g., a vector that modulates the level of expression or activity of the target protein in the cell. The construct can over-express the protein, e.g., from a heterologous promoter. The other aliquot of cells can either be unmodified or can be modified with a suitable control or reference vector (e.g., a vector lacking an insert).

The array of available recombinant methods includes numerous other techniques. For example, expression of a target protein can be modulated by modifying the regulatory region of the gene that encodes it. Transcription of the gene can be altered by: altering the regulatory sequences of the endogenous gene, e.g., by the addition of regulatory sequence, such as a DNA-binding site for a transcriptional repressor or activator, or by the removal of a regulatory sequence, such as an enhancer or a DNA-binding site for a transcriptional activator or repressor. Endogenous genes can be modified by targetted gene recombination or random integration, e.g., to insert an ectopic promoter.

Expression constructs can be designed to delete all or part of a coding sequence of a gene, such that the encoded protein is not produced, is non-functional, is activated, or has a dominant negative activity. A construct can be designed to replace all or part of a coding sequence of a gene, e.g., with a sequence that codes for an altered variant of the protein, e.g., an activated or dominant negative variant.

A construct can be introduced to a chromosome by homologous recombination or located on an extrachromosomal element, e.g., an artificial chromosome. Exemplary methods for creation of vectors and transfection of cells are described in Sambrook et al. (2001) Molecular Cloning: A Laboratory Manual, Cold Spring Harbor Laboratory. In another embodiment, the cell contains a nucleic acid molecule that can bind to a cellular nucleic acid sequence, e.g., mRNA, encoding a protein described herein and can inhibit expression of the protein, e.g., an antisense, siRNA molecule.

The cells can be derived from animals that have altered expression or activity of the target protein. For example, cells can be obtained from a reference animal (e.g., a normal or wild-type animal) and from non-reference animal, e.g., a transgenic animal or an animal with a genetic alteration (e.g., a natural genetic variant).

Examples of transgenic animal include those that are modified to include nucleic acid that increases expression of a target gene, e.g., by introducing of a construct that expresses the target protein (including fragments and variants of the canonical target protein) from a heterologous promoter in one or more cell types or by introduction of a heterologous promoter region into an endogenous gene.

Methods for generating non-human transgenic animals can involve introducing a nucleic acid of interest, e.g. one that decreases or increases the expression of a gene, into the germ line of a non-human animal to make a transgenic animal. Although rodents, e.g., rats, mice, and guinea pigs, are preferred, other non-human animals can be used. For example, one or several copies of the nucleic acid of interest may be incorporated into the DNA of a mammalian embryo by standard transgenic techniques (see, e.g., Nagy et al. Manipulating the Mouse Embryo: A Laboratory Manual (3rd ed. 2003)). A protocol for the production of a transgenic rat can be found in Bader et al. (1996) Clin. Exp. Pharmacol. Physiol. Suppl. 3:S81-87.

Transgenic mice containing a promoter-transgene may be generated by established methods. The transgene can include regulatory sequences that direct expression of a gene to a specific cell type, e.g., a skin cell, pancreatic cell, adipose cell, nerve cell, or other.

Primary cells can be, e.g., fibroblast cells, e.g., skin fibroblasts or mouse embryonic fibroblasts (MEFs). For example, Sirt1 −/− cells, e.g., fibroblasts, e.g., MEFs, can be derived from Sirt1 null (−/−) mice (McBurney et al. (2003) Mol. Cell. Biol. 23:38-54), or IGF-IR −/− or IGF-1R +/− cells, e.g., fibroblasts, e.g., MEFs, can be derived from IGF-1R knockout mice (Holzenberger et al. (2003) Nature 421:182-7).

Artificial transcription factors can also be used to regulate the expression of genes encoding target proteins. The artificial transcription factor can be designed or selected from a library. The protein can include one or more zinc finger domains. For example, the protein can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19:656; and Wu et al. (1995) Proc. Nat. Acad. Sci. USA 92:344 for, among other things, methods for creating libraries of varied zinc finger domains. Optionally, the zinc finger protein can be fused to a transcriptional regulatory domain, e.g., an activation domain to activate transcription or a repression domain to repress transcription. The zinc finger protein can itself be encoded by a heterologous nucleic acid that is delivered to a cell or the protein itself can be delivered to a cell (see, e.g., U.S. Pat. No. 6,534,261). The heterologous nucleic acid that includes a sequence encoding the zinc finger protein can be operably linked to an inducible promoter, e.g., to enable fine control of the level of the zinc finger protein, and thus the target protein, in the cell.

Screening Parameters

To determine if a compound has a differential effect on a pathway or target protein, a parameter of a cell contacted with the compound can be evaluated. The parameter can related to any qualitative or quantitative property. For example, the parameter can provide an assessment of cell function, behavior, signalling, and so forth.

Exemplary parameters that can be measured in a cell-based assay include parameters indicative of cellular proliferation (e.g., by incorporation of bromodeoxyuridine (BrdU) or radiolabeled thymidine, by reduction of 3-(4, 5-dimethylthiazolyl-2)-2,5-diphenyltetrazolium bromide (MTT), or by metabolite incorporation), apoptosis (e.g., by TUNEL staining), levels of secondary metabolites (e.g., ATP or ADP), uptake of metabolites (e.g., glucose, citrate, malate, or fumarate, e.g., radiolabeled versions thereof), acetylation status of proteins (e.g., nuclear proteins, e.g., histones or transcription factors, e.g., p53, FoxO1, FoxO3, Tat, PPARγ), phosphorylation status of proteins (e.g., IGF-1R or an AMP-K substrate), secretion of extracellular products (e.g., growth factors, cytokines, or hormones, e.g., insulin), gene expression (e.g., using a reporter construct, by northern analysis, or by microarray analysis), and protein expression (e.g., by western analysis, protein microarray analysis).

Still other cell-based assays including contacting cells with the test compound and evaluating resistance to a stress, for example, hypoxia, DNA damage (genotoxic stress), or oxidative stress. For example, it is possible to determine whether hypoxia-mediated cell death is attenuated by the test compound.

The parameter may be an assessment of hormesis, e.g., responsiveness to UV, H₂O₂, or adiramycin.

The parameter may also relate to a property of a cellular component, e.g., a protein, nucleic acid, or metabolite. For example, the parameter can be an assessment of the modification state of a cellular component, e.g., acetylation state of a sirtuin substrate, e.g., a SIRT1 substrate such as a histone or transcription factor. In the case of p53, the presence of an acetyl groups can be found at one or more of: K₃₇₀, K₃₇₁, K₃₇₂, K₃₈₁, and/or K₃₈₂ of the p53 sequence. Acetylation state can be evaluated, e.g., by mass spectrometry methods, by using radiolabeled substrates, e.g., comprising ³H, ¹⁴C, or by using antibodies specific for acetylated or deacetylated forms of the substrate.

The parameter can be an assessment of the level of a metabolite (e.g., a carboxylate) in a cell. The cell can be maintained under conditions that facilitate evaluating uptake or egress of the metabolite. Accordingly, the parameter can be an indicator of activity of a transporter, e.g., an INDY transporter.

The parameter can be an assessment of the phosphorylation state of a cellular component, e.g., a cell surface receptor, a kinase, or enzyme. Exemplary components include IGF-1H or one or more AMP-K substrates, e.g., acetyl CoA carboxylase, glycogen synthase, or insulin receptor substrate-1 (IRS1). The phosphorylation state can refer to the presence or absence of one or more phosphoryl groups at one or more tyrosine (Y), serine (S), or threonine (T) residues of a cellular component, e.g., IGF-1H, acetyl CoA carboxylase, glycogen synthase, or IRS1. For example, IGF1-R can be phosphorylated on one or more tyrosine residues, e.g., Y₁₁₃₁, Y₁₁₃₅, and/or Y₁₁₃₆ of the mature human IGF1-R. Phosphorylation status can be determined, e.g., by mass spectrometry methods, by using radiolabeled substrates, e.g., comprising ³²P or ³³P, or by using antibodies specific for phosphorylated or dephosphorylated forms of the substrate.

The parameter can be an assessment of secretion of a cellular component, e.g., a hormone or growth factor, e.g., insulin. Secretion can be measured directly, e.g., by immunological methods, e.g., by ELISA. Secreted products can also be measured indirectly, e.g., by measuring an activity the product exerts on a substrate, e.g., an enzymatic activity, or by measuring an activity the product exerts on another cell, e.g., a signaling activity, or using a secreted reporter gene.

The parameter can be an assessment of a gene, e.g., gene regulated by a pathway or target protein. Gene expression can be evaluated by a variety of methods, including a reporter genes, RT-PCR, Northerns, and microarrays.

Reporter genes of promoters regulated by pathways that involve homologs of SIR2, Indy, AMP-K, or IGF-1R can be made by operably linking a regulatory sequence to a sequence encoding a reporter gene. A number of methods are available for designing reporter genes. For example, the sequence encoding the reporter protein can be linked in frame to all or part of the sequence that is normally regulated by the regulatory sequence. Such constructs can be referred to as translational fusions. It is also possible to link the sequence encoding the reporter protein to only regulatory sequences, e.g., the 5′ untranslated region, TATA box, and/or sequences upstream of the mRNA start site. Such constructs can be referred to as transcriptional fusions. Still other reporter genes can be constructed by inserting one or more copies (e.g., a multimer of three, four, or six copies) of a regulatory sequence into a neutral or characterized promoter. See, e.g., U.S. Ser. No. 60/614,146.

Exemplary reporter proteins include chloramphenicol acetyltransferase, green fluorescent protein and other fluorescent proteins (e.g., artificial variants of GFP), beta-lactamase, beta-galactosidase, luciferase, and so forth. The reporter protein can be any protein other than the protein encoded by the endogenous gene that is subject to analysis. Epitope tags can also be used.

Expression of a gene can be evaluated by detecting an mRNA, e.g., the transcript from the gene of interest or detecting a protein, e.g., the protein encoded by the gene of interest. Exemplary methods for evaluating mRNAs include northern analysis, RT-PCR, microarray hybridization, SAGE, differential display, and monitoring reporter genes. Exemplary methods for evaluating proteins include immunoassays (e.g., ELISAs, immunoprecipitations, westerns), 2D-gel electrophoresis, and mass spectroscopy. It is possible to evaluate fewer than 100, e.g., less than 20, 10, 5, 4 or 3 different molecular species, e.g., to only evaluate the expression of the gene of interest, although it is typically useful to include at least one or two controls (e.g., a house keeping gene). It is also possible to evaluate multiple molecular species, e.g., in parallel, e.g., at least 10, 50, 20, 100, or more different species. See, e.g., the usage of microarrays, e.g. as described below.

One method for comparing transcripts uses nucleic acid microarrays that include a plurality of addresses, each address having a probe specific for a particular transcript. Such arrays can include at least 100, or 1000, or 5000 different probes, so that a substantial fraction, e.g., at least 10, 25, 50, or 75% of the genes in an organism are evaluated. mRNA can be isolated from a cell or other sample of the organism. The mRNA can be reversed transcribed into labeled cDNA. The labeled cDNAs are hybridized to the nucleic acid microarrays. The arrays are detected to quantitate the amount of cDNA that hybridizes to each probe, thus providing information about the level of each transcript.

Methods for making and using nucleic acid microarrays are well known. For example, nucleic acid arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145). The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the nucleic acid. The Tₘ of the capture probe can be optimized by prudent selection of the complementarity region and length. Ideally, the Tₘ of all capture probes on the array is similar, e.g., within 20, 10, 5, 3, or 2° C. of one another. A database scan of available sequence information for a species can be used to determine potential cross-hybridization and specificity problems.

The isolated mRNA from samples for comparison can be reversed transcribed and optionally amplified, e.g., by rtPCR, e.g., as described in (U.S. Pat. No. 4,683,202). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array. A profile that includes information about the expression of a plurality of different genes can be developed, e.g., for each cell being evaluated.

Other methods for quantitating mRNAs include: quantitative RT-PCR. In addition, two nucleic acid populations can be compared at the molecular level, e.g., using subtractive hybridization or differential display to evaluate differences in mRNA expression, e.g., between the two cells that have different expression or activity of the target protein.

Target Proteins

A variety of pathways and target proteins can be used to evaluate compounds. One example of a target protein is a sirtuin. The term “sirtuin” and “sirtuin” include amino acids sequences that have a SIR2 domain or a fragment thereof (the fragment need not also include the SIR2 domain). The fragments have at least one function of a sirtuin protein or are folded. Functional fragments can, for example, have deacetylase activity or interact with a sirtuin binding partner, e.g., PPAR-gamma, PGC1, or NcoR. A sirtuin can be encoded using a nucleic acid that includes artificially chosen codons. Sirtuins include proteins that are scored as hits in the Pfam family for SIR2 domains.

To identify the presence of a “SIR2” domain in a protein sequence, and make the determination that a polypeptide or protein of interest has a particular profile, the amino acid sequence of the protein can be searched against the Pfam database of HMMs (e.g., the Pfam database, release 2.1) using the default parameters (available from the Sanger web site: www-sanger-ac-uk/Software/Pfam/HMM_search). The SIR2 domain is indexed in Pfam as entry number PF02146 and in INTERPRO as INTERPRO description (entry IPR003000). At present PF02146 includes 168 sequences. SIR2 domains can have a fold that is structurally similar to PDB entry 1ICI or 1M2H.

For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314. For example, closely related homologs of human SIRT1 (NP_{—}036370.2) in M. musculus (NP_{—}062786.1; 737 amino acid) and R. norvegicus (XP_{—}228146.2; 700 amino acid). Additional homologs include B. taurus Bt.13818; C. elegans Cel.12479; C. intestinalis Cin.7948; C. intestinalis Cin. 13319; D. rerio Dr.10536; D. melanogaster Dm.415; G. gallus Gga.11206; M. musculus Mm.150679; M. musculus Mm.348981 ; M. musculus Mm.348984; R. norvegicus Rn.42098; X. laevis X1.8444; and X. tropicalis Str.10623.

Human sirtuins include, e.g., the following amino acids sequences: human sirtuin 1 (GenBank Accession No: NP_{—}036370.2); human sirtuin 2 isoform 1 (GenBank Accession No: NP_{—}036369.2); human sirtuin 2 isoform 2 (GenBank Accession No: NP_{—}085096.1); human sirtuin 3 (GenBank Accession No: NP_{—}036371.1); human sirtuin 4 (GenBank Accession No: NP_{—}036372.1); human sirtuin 5 isoform 1 (GenBank Accession No: NP_{—}036373.1); human sirtuin 5 isoform 2 (GenBank Accession No: NP_{—}112534.1); human sirtuin 6 (GenBank Accession No: NP_{—}057623.1); and human sirtuin 7 (GenBank Accession No: NP_{—}057622.1). With respect to any embodiment described herein for SIRT1, the embodiment can be adapted and implemented using another sirtuin, e.g., SIRT2, SIRT3, SIRT4, SIRT5, SIRT6, or SIRT7.

The terms “sirtuin nucleic acid” and “mammalian homolog of a SIR2 gene” includes all nucleic acids sequences that encode sirtuin proteins, and all nucleotide sequences that are complementary or fragments thereof that encode functional or folded fragments of a sirtuin protein. Exemplary sirtuin nucleic acids include human Sir2 SIRT1 mRNA (GenBank Accession No. AF083106); mouse SIRT1 mRNA (GenBank Accession No: AF214646); rat SIRT1 mRNA (GenBank Accession No: XM_{—}228146); human Sir2 SIRT2 mRNA (GenBank Accession No: AF083107); mouse Sir2 SIRT2 mRNA (GenBank Accession No: AF299337); human Sir2 SIRT3 mRNA (GenBank Accession No: AF083108); mouse Sir2 SIRT3 mRNA splice variants (GenBank Accession Nos: AF299339 and AF 299338); human Sir2 SIRT4 mRNA (GenBank Accession No: AF083109); human Sir2 SIRT5 mRNA (GenBank Accession No: AF083110); human Sir2 SIRT6 mRNA (GenBank Accession No: AF233396); mouse Sir2 SIRT6 mRNA (GenBank Accession No: NM_{—}181586); human Sir2 SIRT7 mRNA (GenBank Accession No: AF233395); mouse Sir2 SIRT7 mRNA (GenBank Accession No: NM_{—}153056), as well as all genomic DNA, cDNA, and synthetic DNA sequences that correspond to, or are complementary or are fragments, e.g., encoding functional protein fragments of the aforementioned nucleic acid sequences.

In Saccharomyces cerevisiae, additional copies of the SIR2 gene increase lifespan (Tissenbaum and Guarente (2001) Nature 410:227-30). An exemplary human homolog of SIR2 is SirT1, Accession No. NP_{—}036370; an exemplary mouse homolog is mSir2α, Accession No. NP_{—}062786.

Mutations in the Indy (I'm not dead yet) gene of Drosophila melanogaster result in a near doubling of the adult lifespan without decrease in fertility or physical activity (Rogina et al. (2000) Science 290:2137-40). An exemplary human homolog is SLC13A2, Accession No. NP_{—}003975; an exemplary mouse homolog is S1c13a2, Accession No. NP_{—}071856.

AMP-activated protein kinase (AMP-K) is a sensor of cell energy status and has been shown to be involved in cellular senescence. An exemplary human AMP-K gene is PRKAA2, Accession No. NP_{—}006243; an exemplary mouse AMP-K gene is Prkaa2, Accession No. NP_{—}835279. The AMPK pathway is defined and described, e.g., in PCT/US03/38628. AMPK Pathway members include AMPK activating proteins, such as AMPKK, and AMPK suppressing proteins such as protein phosphatase 2C, AMPK direct targets, and AMPK indirect targets.

Still other pathway members include AMPK substrates. For example, in liver cells, acetyl-CoA carboxylase (ACC) and 3-hydroxyl-3-methylglutaryl-CoA reductase (HMGR) are AMPK substrates. AMPK activation causes HMGR phosphorylation and inhibition of fatty acid and sterol synthesis. Additional exemplary substrates include malonyl-Co decarboxylase (MCD) and nitric oxide synthetase (NOS).

The target protein can be a component of the GH pathway. The GH pathway is defined and described, e.g., in U.S. Ser. No. 10/656,530. The GH/IGF-1 axis includes a series of extracellular and intracellular signalling components that have as a downstream target, the transcription factor Forkhead. Major components of the GH/IGF-1 axis are shown in FIG. 2. The components can be divided into three categories: pre-IGF-1, IGF-1, and post-IGF-1 components. “Pre-IGF-1 components” include GH, GHS, GHS-R, GHRH, GHRH-R, SST, and SSTR. “Post-IGF-1 components” include IGF-1-R and intracellular signalling components including PI(3) kinase, PTEN phosphatase, PI(3,4)P₂, 14-3-3 protein, and PI(3,4,5)P₃ phosphatidyl inositol kinases, AKT serine/threonine kinase (e.g., AKT-1, AKT-2, or AKT-3), or a Forkhead transcription factor (such as FOXO- 1, FOXO-3, or FOXO-4). Antagonism of the insulin-like growth factor (IGF-1) pathway in Caenorhabditis elegans and D. melanogaster leads to an extension of lifespan (Kenyon et al. (1993) Nature 366:461-4; Tatar et al. (2001) Science 292:107-10). C. elegans daf-2 encodes an insulin-like growth factor receptor (IGF-1R). An exemplary human homolog is IGF1R, Accession No. NP_{—}000866; an exemplary mouse homolog is Igf1r, Accession No. NP_{—}034643.

Additional Assays

In addition to evaluating a compound for its effect on cells that have different expression or activity of the target protein, the compound can be evaluated using additional assays, including in vitro assays, in vivo assays, as well as other cellular assays. The compound can be evaluated in such additional assays before, during, or after performing the cellular assays.

Examples of in vitro assays include evaluating the effect of a compound on an activity, e.g., a binding activity or an enzymatic activity, of the target protein (or a fragment thereof, e.g., a functional fragment thereof).

For example, the effect of a compound on an activity of a sirtuin can be further evaluated in vitro, e.g., using a method described herein (e.g., hereinabove) or another method. The compound can be contacted to a sirtuin or a fragment thereof in the presence of a substrate and cofactors (such as NAD). Exemplary substrates for Sirt1 acetyltransferase activity include FOXO3a, p53, PPARγ, Tat, and histones, including acetylated peptides from such substrates. The substrate can be a single amino acid (e.g., an acetylated lysine), a peptide (e.g., a N-terminal peptide of a histone, or an acetylated p53 peptide), or a protein. An acetylated substrate can include a fluorophore, e.g., which can be used to monitor the acetylation states of the substrate. The FLUOR-DE-LYS™ substrate from BIOMOL (Plymouth Meeting, Pa.) includes one such exemplary modification. Examples of sirtuin assays are also described in US 2003-0207325 and US 2004-0005574.

Some exemplary screening assays for assessing activity or function of Sirt1 include one or more of the following features: use of a transgenic cell, e.g., with a transgene encoding Sirt1 or a mutant thereof; use of a mammalian cell that expresses Sirt1; use of an enzymatic assay for Sirt1, e.g., to evaluate deacetylation of a substrate, e.g., an amino acid, a peptide or a protein; detection of binding to Sirt1, e.g., by a Sirt1 binding partner or a test compound, for example, where the compound is, for example, a peptide, protein, antibody or small organic molecule; e.g., the compound modulates (e.g., stimulates or inhibits) an interaction between Sirt1 and a Sirt1-binding partner; use of proximity assays that detect interaction between a Sirt1 and a Sirt1-binding partner, e.g., a protein, e.g., a mitochondrial or nuclear protein, e.g., a histone or transcription factor (e.g., p53 or Tat), or fragments thereof, for example, fluorescence proximity assays; use of radio-labeled substrates, e.g. ³⁵S, ³H, ¹⁴C, e.g., to determine acetylation status, metabolic status, and so forth; and use of antibodies specific for certain acetylated or de-acetylated forms of the substrate.

Some exemplary screening assays for assessing activity or function of NaCT or other transporter (e.g., an INDY transporter) include one or more of the following features: use of a transgenic cell, e.g., with a transgene encoding NaCT or a mutant thereof; use of a mammalian cell that expresses NaCT; use of an functional assay for NaCT, e.g., uptake of a substrate, e.g., citrate, malate or fumarate, e.g., radiolabeled versions thereof; detection of uptake by NaCT of a substrate, e.g., citrate, malate, or fumarate, in the presence of a test compound; detection of binding to NaCT, e.g., by a test compound, for example, where the compound is, for example, a peptide, protein, antibody or small organic molecule. Exemplary assays for NaCT activity are described in WO 2004/043925.

The effect of a compound on an activity of AMP-K can be further evaluated. AMP-K can phosphorylate proteins in the presence of adenosine monophosphate, e.g., in vitro. Some exemplary assays for assessing activity or function of AMP-K include one or more of the following features: use of a transgenic cell, e.g., with a transgene encoding AMP-K or a mutant thereof; use of a mammalian cell that expresses AMP-K; use of an enzymatic assay for AMP-K, e.g., to evaluate phosphorylation of a substrate, e.g., an amino acid, a peptide or a protein, e.g., acetyl CoA carboxylase, glycogen synthase, or IRS1; detection of binding to AMP-K, e.g., by an AMP-K binding partner or a test compound, for example, where the compound is, for example, a peptide, protein, antibody or small organic molecule, e.g., the compound modulates (e.g., stimulates or inhibits) an interaction between AMP-K and an AMP-K binding partner; use of proximity assays that detect interaction between a AMP-K and an AMP-K binding partner, e.g., a protein or fragment thereof, for example, fluorescence proximity assays; use of radio-labeled substrates, e.g. ³²P, ³³P, e.g., to determine phosphorylation status; and use of antibodies specific for certain phosphorylated or dephosphorylated forms of the substrate. An exemplary commercially available assay for AMP kinase activity is the HITHUNTER™ Kinase Toolbox (DiscoveRx, Fremont, Calif.). Exemplary AMP kinase assays are also described in WO 02/09726.

The effect of a compound on an activity of IGF-1R can be further evaluated. IGF-1R is a receptor tyrosine kinase. IGF1-R can be phosphorylated on one or more tyrosine residues, e.g., Y₁₁₃₁ Y₁₁₃₅ Y₁₁₃₆, of the mature human IGF1-R. Some exemplary assays for assessing activity or function of IGF-1R include one or more of the following features: use of a transgenic cell, e.g., with a transgene encoding IGF-1R or a mutant thereof, e.g., a constitutively active IGF-1R, or a chimeric fusion protein comprising a domain of IGF-1R; use of a mammalian cell that expresses IGF-1R; use of an enzymatic assay for IGF-1R, e.g., to evaluate phosphorylation of a substrate, e.g., autophosphorylation; detection of binding to IGF-1R, e.g., by an IGF-1R binding partner or a test compound, for example, where the compound is, for example, a peptide, protein, antibody or small organic molecule, e.g., the compound modulates (e.g., stimulates or inhibits) an interaction between IGF-1R and an IGF-1R-binding partner; use of proximity assays that detect interaction between an IGF-1R and an IGF-1R-binding partner, e.g., a protein or a fragment thereof, for example, fluorescence proximity assays; use of radio-labeled substrates, e.g. ³²P, ³³P, e.g., to determine phosphorylation status; and use of antibodies (e.g., polyclonal or monoclonal antibodies) specific for certain phosphorylated or de-phosphorylated forms of the substrate.

Antibodies

Immunoglobulins can be produced that bind to a protein described herein or a binding partner of a protein described herein. Such immunoglobulins can be used as test compounds in the method described herein or as reagents to facilitate an assay. In one embodiment, the immunoglobulin is human, humanized, deimmunized, or otherwise non-antigenic in the subject.

An immunoglobulin can be, for example, an antibody or an antigen-binding fragment thereof. As used herein, the term “immunoglobulin” refers to a protein consisting of one or more polypeptides that include one or more immunoglobulin variable domain sequences. A typical immunoglobulin includes at least a heavy chain immunoglobulin variable domain and a light chain immunoglobulin variable domain. An immunoglobulin protein can be encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH₂-terminus (about 110 amino acids) and a kappa or lambda constant region gene at the COOH-terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids). The term “antigen-binding fragment” of an antibody (or simply “antibody portion” or “fragment”), as used herein, refers to one or more fragments of a full-length antibody that retain the ability to specifically bind to the antigen. Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques, and the fragments are screened for utility in the same manner as are intact antibodies.

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey). Preferably, the non-human antibody is a rodent (mouse or rat antibody). Method of producing rodent antibodies are known in the art. Non-human antibodies can be modified, e.g., humanized or deimmunized. Human monoclonal antibodies can be generated using transgenic mice carrying the human immunoglobulin genes rather than the mouse system (see, e.g., WO 91/00906 and WO 92/03918). Other methods for generating immunoglobulin ligands include phage display (e.g., as described in U.S. Pat. No. 5,223,409 and WO 92/20791).

Administration

An agent, e.g., an agent that includes or is based on a compound identified or evaluated by a methods described herein, can be incorporated into a pharmaceutical composition, e.g., a composition that includes a pharmaceutically acceptable carrier. Examples of pharmaceutically acceptable carriers include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition can be formulated to be compatible with an intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods disclosed herein, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

A therapeutically effective amount of protein or polypeptide (e.g., an effective dosage) includes ranges that can be identified using standard methods. In some cases, the amount will be, e.g., from about 0.001 to 30 mg/kg body weight, preferably about 0.01 to 25 mg/kg body weight. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For topical application, the compositions disclosed herein can include a medium compatible with skin. Such topical pharmaceutical compositions can exist in many forms, e.g., in the form of a solution, cream, ointment, gel, lotion, shampoo, or aerosol formulation adapted for application to the skin. A wide variety of carrier materials can be employed in the compositions disclosed herein such as alcohols, aloe vera gel, allantoin, glycerine, vitamin A and E oils, mineral oils, and polyethylene glycols. Other additives, e.g., preservatives, fragrance, sunscreen, or other cosmetic ingredients, can be present in the composition. The topical composition can be applied and removed immediately, or it can be applied and left on the skin surface, e.g., the face, for an extended period of time, e.g., overnight or throughout the day.

Methods of Treatment

Compounds identified or evaluated by a method described herein can be used to prevent, treat or otherwise ameliorate cancer, diabetes, obesity, frailty, skin aging, and neurodegenerative disorders, among other disorders associated with these pathways or target proteins. Examples of neurodegenerative disorders include Alzheimer's, Parkinson's, and polyglutamine aggregation disorders (e.g., Huntington's disease).

Aged Skin

Signs of aged skin include, e.g., wrinkles, lines, sagging, freckles, tanned skin, discoloration, hyperpigmentation, age spots, e.g., “liver spots”, thinning of the skin, cataracts, epidermal hyperplasia, skin elastosis, degradation of extracellular matrix, or precancerous or cancerous skin growths (actinic keratoses, solar keratoses).

A compound can also be tested for its affect on skin, e.g., using an animal model. Exemplary assays for evaluating skin include those described in US 20040110203.

Cancer

Examples of cancerous disorders include, but are not limited to, solid tumors, soft tissue tumors, and metastatic lesions. As used herein, the term “cancer” is meant to include all types of cancerous growths or oncogenic processes, metastatic tissues or malignantly transformed cells, tissues, or organs, irrespective of histopathologic type or stage of invasiveness. The cancer may be a malignant or non-malignant cancer. In some embodiments, the methods prevent or treat tumor proliferation and/or metastasis.

Cancers or tumors include, but are not limited, to biliary tract cancer; brain cancer; breast cancer; cervical cancer; choriocarcinoma; colon cancer; endometrial cancer; esophageal cancer; gastric cancer; intraepithelial neoplasms; leukemias, lymphomas; liver cancer; lung cancer (e.g. small cell and non-small cell); melanoma; neuroblastomas; oral cancer; ovarian cancer; pancreatic cancer; prostate cancer; rectal cancer; sarcomas; skin cancer; testicular cancer; thyroid cancer; and renal cancer, as well as other carcinomas and sarcomas. Examples of tumors and cancers which are p53 dependent include colon cancer, breast cancer, lung cancer, bladder cancer, brain cancer, pancreatic cancer, stomach cancer, esophageal cancer, sarcomas, cervical cancer, liver cancer, lymphomas and neuroblastomas. A discussion of p53 dependent cancers are discussed, e.g., in Vogelstein et al. (2000) Nature 408:307.

Metastatic lesions of the aforementioned cancers can also be treated or prevented using a compound identified by the methods described herein.

Animal models for cancer and neoplasia are routine in the art and include xenografts in nude mice. See, e.g., Kobayashi et al., Int. J. Canc., 57: 727-733d (1994); Rabbani et al., Int. J. Cancer 63: 840-845 (1995); and Xing et al., Canc. Res., 57: 3585-3593 (1997).

Diabetes

Examples of diabetes include insulin dependent diabetes mellitus and non-insulin dependent diabetes. In some instances, a patient can be identified as being at risk of developing diabetes by having impaired glucose tolerance (IGT), or fasting hyperglycemia.

Insulin dependent diabetes mellitus (Type 1 diabetes) is an autoimmune disease, where insulitis leads to the destruction of pancreatic β-cells. At the time of clinical onset of type 1 diabetes mellitus, significant number of insulin producing β cells are destroyed and only 15% to 40% are still capable of insulin production (McCulloch et al. (1991) Diabetes 40:673-679). b-cell failure results in a life long dependence on daily insulin injections and exposure to the acute and late complication of the disease.

Type 2 diabetes mellitus is a metabolic disease of impaired glucose homeostasis characterized by hyperglycemia, or high blood sugar, as a result of defective insulin action which manifests as insulin resistance, defective insulin secretion, or both. A patient with Type 2 diabetes mellitus has abnormal carbohydrate, lipid, and protein metabolism associated with insulin resistance and/or impaired insulin secretion. The disease leads to pancreatic beta cell destruction and eventually absolute insulin deficiency. Without insulin, high glucose levels remain in the blood. The long term effects of high blood glucose include blindness, renal failure, and poor blood circulation to these areas, which can lead to foot and ankle amputations. Early detection is critical in preventing patients from reaching this severity. The majority of patients with diabetes have the non-insulin dependent form of diabetes, currently referred to as Type 2 diabetes mellitus.

This disclosure also includes methods of treating disorders related to or resulting from diabetes, for example end organ damage, diabetic gastroparesis, diabetic neuropathy, cardiac dysrythmia, etc.

Examples of animal models of diabetes include: the Wistar Fatty Rat; the Zucker Diabetic Fatty (ZDF) Rat; the Goto-Kakizaki Rat; the Olete Rat; the Obese Spontaneously Hypertensive Rat (Shrob, Koletsky Rat): A Model of Metabolic Syndrome X ; the Neonatally Streptozocin-Induced (N-STZ) Diabetic Rats; the NSY Mouse: An Animal Model of Human Type 2Diabetes Mellitus with Polygenic Inheritance; and the New Zealand Obese Mouse: A Polygenic Model of Type 2 Diabetes. See, e.g., “Animal Models of Diabetes,” Sina et al. (ISBN: 9058230961) Harwood Academic 2000.

Obesity and the Metabolic Syndrome

A compound identified by a method described herein can be used to modulate a fat cell, e.g., an adipocyte, e.g., differentiation of the adipocyte. For example, a compound described herein can be administered in an amount effective to prevent fat accumulation in a normal or a pathological state. Disorders relating to adipocytes include obesity. “Obesity” refers to a condition in which a subject has a body mass index of greater than or equal to 30. “Over-weight” refers to a condition in which a subject has a body mass index of greater or equal to 25.0. The body mass index and other definitions are according to the “NIH Clinical Guidelines on the Identification and Evaluation, and Treatment of Overweight and Obesity in Adults” (1998). In particular, obesity can lead to type II diabetes in successive phases. Clinically, these phases can be characterized as normal glucose tolerance, impaired glucose tolerance, hyperinsulinemic diabetes, and hypoinsulinemic diabetes. Such a progressive impairment of glucose storage correlates with a rise in basal glycemia.

A compound identified by a method described herein can be used to treat or prevent other metabolic disorders too, e.g., a metabolic syndrome. Metabolic syndrome (e.g., Syndrome X) is characterized by a group of metabolic risk factors in one person. They include: central obesity (excessive fat tissue in and around the abdomen), atherogenic dyslipidemia (blood fat disorders—mainly high triglycerides and low HDL cholesterol—that foster plaque buildups in artery walls); insulin resistance or glucose intolerance (the body can't properly use insulin or blood sugar); prothrombotic state (e.g., high fibrinogen or plasminogen activator inhibitor [−1] in the blood); raised blood pressure (i.e., hypertension) (130/85 mmHg or higher); and proinflammatory state (e.g., elevated high-sensitivity C-reactive protein in the blood). The underlying causes of this syndrome can include overweight/obesity, physical inactivity and genetic factors. People with metabolic syndrome are at increased risk of coronary heart disease, other diseases related to plaque buildups in artery walls (e.g., stroke and peripheral vascular disease) and type 2 diabetes. Metabolic syndrome is closely associated with a generalized metabolic disorder called insulin resistance, in which the body is unable to insulin efficiently.

Exemplary models for the treatment of obesity include two primary animal model systems: 1) diet-induced obesity (DIO) caused by feeding rodents ˜60% fat content of caloric intake. Animals treated for up to 12-16 weeks on this type of diet gain substantial body weight (>50% increase), accumulate excessive fat mass, become hyperglycemic, hyperinsulinemic and insulin resistant. In this model compounds can be tested prior to the initiation of the diet or at any time during development of obesity. 2) db/db mutant mice (leptin receptor spontaneous mutant). These animals exhibit a similar phenotype as the DIO animals only more severe with regard to various readouts. Animals can be treated similar to the DIO model. As a surrogate readout of SirT1 inhibitor activity, sister animals can be sacrificed along the treatment regimen and assessed biochemically for increased acetylation status of FoxO1 proteins in various tissues, such as liver, muscle and white adipose tissue.

Alzheimer's Disease

Alzheimer's disease (AD) is a complex neurodegenerative disease that results in the irreversible loss of neurons and is an example of a neurodegenerative disease that has symptoms caused at least in part by protein aggregation. A compound identified by the methods described herein can be used to ameliorate at least one symptom of a subject that has AD. Clinical hallmarks of Alzheimer's Disease include progressive impairment in memory, judgment, orientation to physical surroundings, and language. Neuropathological hallmarks of AD include region-specific neuronal loss, amyloid plaques, and neurofibrillary tangles.

Amyloid plaques are extracellular plaques containing the β amyloid peptide (also known as Aβ, or Aβ42), which is a cleavage product of the β-amyloid precursor protein (also known as APP). Neurofibrillary tangles are insoluble intracellular aggregates composed of filaments of the abnormally hyperphosphorylated microtubule-associated protein, tau. Amyloid plaques and neurofibrillary tangles may contribute to secondary events that lead to neuronal loss by apoptosis (Clark and Karlawish, Ann. Intern. Med. 138(5):400-410 (2003)). For example, β-amyloid induces caspase-2-dependent apoptosis in cultured neurons (Troy et al. J. Neurosci. 20(4):1386-1392). The deposition of plaques in vivo may trigger apoptosis of proximal neurons in a similar manner.

In one embodiment, a non-human animal model of AD (e.g., a mouse model) is used, e.g., to evaluate a compound or a therapeutic regimen, e.g., of a compound described herein. For example, U.S. Pat. No. 6,509,515 describes one such model animal which is naturally able to be used with learning and memory tests. The animal expresses an amyloid precursor protein (APP) sequence at a level in brain tissues such that the animal develops a progressive neurologic disorder within a short period of time from birth, generally within a year from birth, preferably within 2 to 6 months, from birth. The APP protein sequence is introduced into the animal, or an ancestor of the animal, at an embryonic stage, preferably the one cell, or fertilized oocyte, stage, and generally not later than about the 8-cell stage. The zygote or embryo is then developed to term in a pseudo-pregnant foster female. The amyloid precursor protein genes are introduced into an animal embryo so as to be chromosomally incorporated in a state which results in super-endogenous expression of the amyloid precursor protein and the development of a progressive neurologic disease in the cortico-limbic areas of the brain, areas of the brain which are prominently affected in progressive neurologic disease states such as AD. The gliosis and clinical manifestations in affected transgenic animals model neurologic disease. The progressive aspects of the neurologic disease are characterized by diminished exploratory and/or locomotor behavior and diminished 2-deoxyglucose uptake/utilization and hypertrophic gliosis in the cortico-limbic regions of the brain. Further, the changes that are seen are similar to those that are seen in some aging animals. Other animal models are also described in U.S. Pat. Nos. 5,387,742; 5,877,399; 6,358,752; and 6,187,992.

Parkinson's Disease

Parkinson's disease includes neurodegeneration of dopaminergic neurons in the substantia nigra resulting in the degeneration of the nigrostriatal dopamine system that regulates motor function. This pathology, in turn, leads to motor dysfunctions. (see, e.g., and Lotharius et al., Nat. Rev. Neurosci., 3:932-42 (2002).) Exemplary motor symptoms include: akinesia, stooped posture, gait difficulty, postural instability, catalepsy, muscle rigidity, and tremor. Exemplary non-motor symptoms include: depression, lack of motivation, passivity, dementia and gastrointestinal dysfunction (see, e.g., Fahn, Ann. N.Y. Acad. Sci., 991:1-14 (2003) and Pfeiffer, Lancet Neurol., 2:107-16 (2003)) Parkinson's has been observed in 0.5 to 1 percent of persons 65 to 69 years of age and 1 to 3 percent among persons 80 years of age and older. (see, e.g., Nussbaum et al., N. Engl. J. Med., 348:1356-64 (2003)). A compound or library of compounds described herein can be evaluated in a non-human animal model of Parkinson's disease. Exemplary animal models of Parkinson's disease include primates rendered parkinsonian by treatment with the dopaminergic neurotoxin 1-methyl-4 phenyl 1,2,3,6-tetrahydropyridine (MPTP) (see, e.g., US Appl 20030055231 and Wichmann et al., Ann. N.Y. Acad. Sci., 991:199-213 (2003); 6-hydroxydopamine-lesioned rats (e.g., Lab. Anim. Sci.,49:363-71 (1999)); and transgenic invertebrate models (e.g., Lakso et al., J. Neurochem., 86:165-72 (2003) and Link, Mech. Ageing Dev., 122:1639-49 (2001)).

Polyglutamine Aggregation Disorders

There are a number of disorders whose pathologies have been attributed, at least in part, to polyglutamine-based aggregation. These disorders include, for example, Huntington's disease, Spinalbulbar Muscular Atrophy (SBMA or Kennedy's Disease), Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCA7), and Spinocerebellar Ataxia 12 (SCA12).

A variety of cell free assays, cell based assays, and organismal assays are available for evaluating polyglutamine aggregation, e.g., Huntingtin polyglutamine aggregation. Some examples are described, e.g., in U.S. 2003-0109476. An exemplary animal model is the transgenic mouse strain of the R6/2 line (Mangiarini et al. Cell 87: 493-506 (1996)). The R6/2 mice are transgenic Huntington's disease mice, which over-express exon one of the human HD gene (under the control of the endogenous promoter). The exon 1 of the R6/2 human HD gene has an expanded CAG/polyglutamine repeat lengths (150 CAG repeats on average). These mice develop a progressive, ultimately fatal neurological disease with many features of human Huntington's disease.

### EXAMPLES

### Example 1

### Boronate Resin in Filter-Plate Retains NAD but Not Nicotinamide

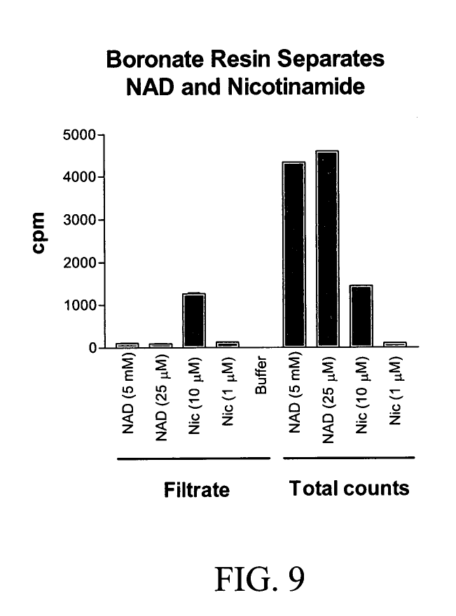

¹⁴C-NAD and ¹⁴C-nicotinamide were separately mixed with boronate resin in a Multiscreen filter plate. Scintillation counting of the filtrate showed that NAD was retained by the resin whereas nicotinamide flowed through in the filtrate. Comparison with the total counts added (filtration through Multiscreen plate alone) showed that up to 5 mM NAD was completely bound by the resin and that nicotinamide was not retained by the resin (FIG. 9).

### Example 2

### Use of a Microplate Filtration Assay to Assay the SIRT Class of Enzymes and Determine Kinetic Parameters

Human SIRT1 deacetylase was assayed as shown in the scheme in FIG. 2. Enzyme was incubated at 37° C. with ¹⁴C-NAD and acetylated peptide [HLKSKKGQSTSRHK(K-Ac)LMFK-OH] (SEQ ID NO:2) in Tris-acetate buffer. After 45 minutes the reaction mixture was diluted with ammonium acetate, pH 9, and transferred to a filter plate containing boronate resin. NAD was retained in the resin while unbound nicotinamide flowed through the filter. Enzyme activity was measured by scintillation counting of the nicotinamide-containing filtrate.

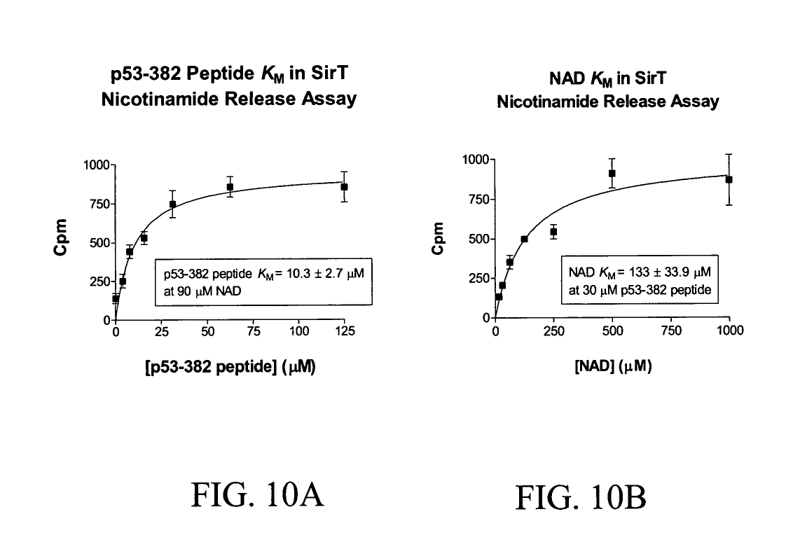

The Michaelis constants (K_{M}) of NAD and the acetylated peptide substrates were determined by measuring enzyme activity in the presence of varied concentrations of each substrate. The results of these assays are depicted in FIG. 10A and FIG. 10B.

### Example 3

### A method to Identify SIRT1 Modulators using MEF Cells

This example describes a method to identify agents that modulate the SIRT1 pathway. The method includes evaluating proliferation of fibroblasts. The method can be used evaluate agents, for example, “cosmeceutical” agents (agents suitable for use in cosmetics).

Mouse embryonic fibroblasts (MEFs) are derived from normal wild-type (Sirt1+/+) mice and Sirt1 knockout (sirt1 −/−) mice by standard methods. A library of compounds is screened for the capacity to modulate (particularly, induce) proliferation of wild-type MEFs. In brief, compounds of the library are contacted to cultures of wild-type MEFs, and cell proliferation is measured as the uptake of ³H-thymidine by the cells of the culture. Several compounds are screened in parallel by performing the assay on a CYTOSTAR-T™ scintillating microplate (Amersham Biosciences, Piscataway, N.J.).

Compounds that induce proliferation in wild-type MEFs are then evaluated for their ability to modulate proliferation in sirt1 −/− MEFs. Compounds that are affect the Sirt1 pathway can, for example, induce proliferation in wild-type MEFs but not in sirt1 −/− MEFs. These compounds are selected as candidate compounds for further analysis or development.

### Example 4

### Exemplary Sirtuin Sequences

TABLE 2
Additional Exemplary sequences

Sirtuin 3 (silent mating type information
regulation 2, homolog) 3; silent mating type
information regulation 2, (S. cerevisiae,
homolog)-like 3; sirtuin 3, Mus musculus,
GenBank ® GI:11967963; Acc.: NP_071878.1

(SEQ ID NO:3)

MVGAGISTPSGIPDFRSPGSGLYSNLQQYDIPYPEAIFELGFFFHNPKPF
FMLAKELYPGHYRPNVTHYFLRLLHDKELLLRLYTQNIDGLERASGIPAS
KLVEAHGTFVTATCTVCRRSFPGEDIWADVMADRVPRCAVCTGVVKPDIV
FFGEQLPARFLLHMADFALADLLLILGTSLEVEPFASLSEAVQKSVPRLL
INRDLVGPFVLSPRRKDVVQLGDVVHGVERLVDLLGWTQELLDLMQRERG
KLDGQDR
Sirtuin 3; sirtuin (silent mating type information
regulation 2, S. cerevisiae, homolog) 3; sirtuin
type 3; sir2-like 3; silent mating type
information regulation 2, S. cerevisiae, homolog
3, Homo sapiens, GenBank ® G1:6912660, Acc.
NP_036371.1

(SEQ ID NO:4)

MAFWGWRAAAALRLWGRVVERVEAGGGVGPFQACGCRLVLGGRDDVSAGL
RGSHGARGEPLDPARPLQRPPRPEVPRAFRRQPRAAAPSFFFSSIKGGRR
SISFSVGASSVVGSGGSSDKGKLSLQDVAELIRARACQRVVVMVGAGIST
PSGIPDFRSPGSGLYSNLQQYDLPYPEAIFELPFFFHNPKPFFTLAKELY
PGNYKPNVTHYFLRLLHDKGLLLRLYTQNIDGLERVSGIPASKLVEAHGT
FASATCTVCQRPFPGEDIRADVMADRVPRCPVCTGVVKPDIVFFGEPLPQ
RFLLHVVDFPMADLLLILGTSLEVEPFASLTEAVRSSVPRLLINRDLVGP
LAWHPRSRDVAQLGDVVHGVESLVELLGWTEEMRDLVQRETGKLDGPDK
Sirtuin type 3, Homo sapiens, GenBank ® 01:5225322,
Acc: AAD40851.1; AF083108_1

(SEQ ID NO:5)

MAFWGWRAAAALRLWGRVVERVEAGGGVGPFQACGCRLVLGGRDDVSAGL
RGSHGARGEPLDPARPLQRPPRPEVPRAFRRQPRAAAPSFFFSSIKGGRR
SISFSVGASSVVGSGGSSDKGKLSLQDVAELIRARACQRVVVMVGAGIST
PSGIPDFRSPGSGLYSNLQQYDLPYPEAIFELPFFFHNPKPFFTLAKELY
PGNYKPNVTHYFLRLLHDKGLLLRLYTQNIDGLERVSGIPASKLVEAHGT
FASATCTVCQRPFPGEDIRADVMADRVPRCPVCTGVVKPDIVFFGEPLPQ
RFLLHVVDFPMADLLLILGTSLEVEPFASLTEAVRSSVPRLLINRDLVGP
LAWHPRSRDVAQLGDVVHGVESLVELLGWTEEMRDLVQRETGKLDGPDK
Sirtuin 4; sirtuin (silent mating type information
regulation 2, S. cerevisiae, homolog) 4; sirtuin
type 4; silent mating type information regulation
2, S. cerevisiae, homolog 4; sir2-like 4, Homo
sapiens, GenBank ® GI:6912662, Acc: NP_036372.1

(SEQ ID NO:6)

MKMSFALTFRSAKGRWIANPSQPCSKASIGLFVPASPPLDPEKELQRFIT
LSKRLLVMTGAGISTESGIPDYRSEKVGLYARTDRRPIQHGDFVRSAPIR
QRYWARNFVGWPQFSSHQPNPAHWALSTWEKLGKLYWLVTQNVDALHTKA
GSRRLTELHGCMDRVLCLDCGEQTPRGVLQERFQVLNPTWSAEAHGLAPD
GDVFLSEEQVRSFQVPTCVQCGGHLKPDVVFFGDTVNPDKVDFVHKRVKE
ADSLLVVGSSLQVYSGYRFILTAWEKKLPIAILNIGPTRSDDLACLKLNS
RCGELLPLIDPC
Sirtuin type 4, Homo sapiens, GenBank ® GI:5225324,
Acc: AAD40852.1, AF083109_1

(SEQ ID NO:7)

MKMSFALTFRSAKGRWIANPSQPCSKASIGLFVPASPPLDPEKVKELQRF
ITLSKRLLVMTGAGISTESGIPDYRSEKVGLYARTDRRPIQHGDFVRSAP
IRQRYWARNFVGWPQFSSHQPNPAHWALSTWEKLGKLYWLVTQNVDALHT
KAGSRRLTELHGCMDRVLCLDCGEQTPRGVLQERFQVLNPTWSAEAHGLA
PDGDVFLSEEQVRSFQVPTCVQCGGHLKPDVVFFGDTVNPDKVDFVHKRV
KEADSLLVVGSSLQVYSGYRFILTAWEKKLPIAILNIGPTRSDDLACLKL
NSRCGELLPLIDPC
Sirtuin type 2, Homo sapiens, GenBank ®
GI:24474785, Acc: CAD43717.1

(SEQ ID NO:8)

MPLAECPSCRCLSSFRSVDFLRNLFSQTLSLGSQKERLLDELTLEGVARY
MQSERCRRVICLVGAGISTSAGIPDFRSPSTGLYDNLEKYHLPYPEAIFE
ISYFKKHPEPFFALAKELYPGQFKPTICHYFMRLLKDKGLLLRCYTQNID
TLERIAGLEQEDLVEAHGTFYTSHCVSASCRHEYPLSWMKEKIFSEVTLK
CEDCQSLVKPDIVFFGESLPARFFSCMQSDFLKVDLLLVMGTSLQVQPFA
SLISKAPLSTPRLLINKEAGQSDPFLGMIMGLGGGMDFDSKKAYRDVAWL
GECDQGCLALAELLGWKKELEDLVRREHASIDAQSGAGVPNPSTSASPKK
SPPPAKDEARTTEREKPQ
Sirtuin 2 (silent mating type information
regulation 2, homolog) 2; silent mating type
information regulation 2, (S. cerevisiae,
homolog)-like; sirtuin 2, Mus musculus, GenBank ®
GI:11967961, Acc: NP_071877.1

(SEQ ID NO:9)

MAEPDPSDPLETQAGKVQEAQDSDSDTEGGATGGEAEMDFLRNLFTQTLG
LGSQKERLLDELTLEGVTRYMQSERCRKVICLVGAGISTSAGIPDFRSPS
TGLYANLEKYHLPYPEAIFEISYFKKHPEPFFALAKELYPGQFKPTICHY
FIRLLKEKGLLLRCYTQNIDTLERVAGLEPQDLVEAHGTFYTSHCVNTSC
RKEYTMGWMKEKIFSEATPRCEQCQSVVKPDIVFFGENLPSRFFSCMQSD
FSKVDLLIIMGTSLQVQPFASLISKAPLATPRLLINKEKTGQTDPFLGMM
MGLGGGMDFDSKKAYRDVAWLGDCDQGCLALADLLGWKKELEDLVRREHA
NIDAQSGSQAPNPSTTISPGKSPPPAKEAARTKEKEEQQ
SIRT2, Rattus norvegicus, GenBank ® GI:14029137,
Acc: AAK51133.1

(SEQ ID NO:10)

MDFLRNLFSQTLSLGSQKERLLDELTLEGVARYMQSERCRRVICLVGAGI
STSAGIPDFRSPSTGLYDNLEKYHLPYPEAIFEISYFKKHPEPFFALAKE
LYPGQFKPTICHYFMRLLKDKGLLLRCYTQNIDTLERIAGLEQEDLVEAH
GTFYTSHCVSASCRHEYPLSWMKEKIFSEVTPKCEDCQSLVKPDIVFFGE
SLPARFFSCMQSDFLKVDLLLVMGTSLQVQPFASLISKAPLSTPRLLINK
EKAGQSDPFLGMIMGLGGGMDFDSKKAYRDVAWLGECDQGCLALAELLGW
KKELEDLVRREHASIDAQSGAGVPNPSTSASPKKSPPPAKDEARTTEREK
PQ
Sirtuin 5 isoform 2; sir2-like 5; sirtuin type 5;
sirtuin (silent mating type information regulation
2, S. cerevisiae, homolog) 5; silent mating type
information regulation 2, S. cerevisiae, homolog
5, Homo sapiens, GenBank ® GI:13787215,
Acc: NP_112534.1

(SEQ ID NO:11)

MRPLQIVPSRLISQLYCGLKPPASTRNQICLKMARPSSSMADFRKFFAKA
KHIVIISGAGVSAESGVPTFRGAGGYWRKWQAQDLATPLAFAHNPSRVWE
FYHYRREVMGSKEPNAGHRAIAECETRLGKQGRRVVVITQNIDELHRKAG
TKNLLEIHGSLFKTRCTSCGVVAENYKSPICPALSGKGAPEPGTQDASIP
VEKLPRCEEAGCGGLLRPHVVWFGENLDPAILEEVDRELAHCDLCLVVGT
SSVVYPAAMFAPQVAARGVPVAEFNTETTPATNRFSHLISISSLIIIKN
Sirtuin 7; sir2-related protein type 7; sirtuin
type 7; sirtuin (silent mating type information
regulation 2, S. cerevisiae, homolog) 7; silent
mating type information regulation 2, S.
cerevisiae, homolog 7, Homo sapiens, GenBank ®
GI:7706712, Acc: NP_057622.1

(SEQ ID NO:12)

MAAGGLSRSERKAAERVRRLREEQQRERLRQVSRILRKAAAERSAEEGRL
LAESADLVTELQGRSRRREGLKRRQEEVCDDPEELRGKVRELASAVRNAA
KYLVVYTGAGISTAASIPDYRGPNGVWTLLQKGRSVSAADLSEAEPTLTH
MSITRLHEQKLVQHVVSQNCDGLHLRSGLPRTAISELHGNMYIEVCTSCV
PNREYVRVFDVTERTALHRHQTGRTCHKCGTQLRDTIVHFGERGTLGQPL
NWEAATEAASRADTILCLGSSLKVLKKYPRLWCMTKPPSRRPKLYIVNLQ
WTPKDDWAALKLHGKCDDVMRLLMAELGLEIPAYSRWQDPIFSLATPLRA
GEEGSHSRKSLCRSREEAPPGDRGAPLSSAPILGGWFGRGCTKRTKRKKV
T
GenBank ® GI:27717613, Acc:XP_234931.1, similar to
sirtuin (silent mating type information regulation
2, S. cerevisiae, homolog) 6 [Homo sapiens]
[Rattus norvegicus]

(SEQ ID NO:13)

MSVNYAAGLSPYADKGKCGLPEIFDPPEELECKVWELARLMWQSSTVVFH
TGAGISTASGIPDFRGPHGVWTMEERGLAPKFDITFENARPSKTHMALVQ
LERMGFLSFLVSQNVDGLHVRSGFPRDKLAELHGNMFVEECPKCKTQYVR
DTVVGTMGLKATGRLCTVAKARGLRACRGELRDTILDWEDALPDRDLTLA
DEASRTADLSVTLGTSLQIRPSGNLPLATKRRGGRLVIVNLQPTKHVCAS
ALPSAPVSPACPLRRTDCPSLWQDRQADLCIHGYVDEVMCKLMKHLGLEI
PTWDGPRVLEALPPLPRPVAPKAEPPVHLNGSYKPKPDSPVPHRPPKRVK
TEAAAS
GenBank ® GI:27690910, Acc: XP_221204.1 similar to
sirtuin 7; sir2-related protein type 7; sirtuin
type 7; sirtuin (silent mating type information
regulation 2, S. cerevisiae, homolog) 7; silent
mating type information regulation 2, S.
cerevisiae, homolog 7 [Homo sapiens] [Rattus
norvegicus]

(SEQ ID NO:14)

MAAGGGLSRSERKAAERVRRLREEQQRERLRQVSRILRKAAAERSAEEGR
LLAESEDLVTELQGRSRRREGLKRRQEEASRGQRVCDDPEELRRKVRELA
GAVRSARHLVVYTGAGISTAASIPDYRGPNGVWTLLQKGRPVSAADLSEA
EPTLTHMSITQLHKHKLGLPRTAISELHGNMYIEVSSAQRTQGLGDKQMS
LTVPSLPQVCTSCIPNREYVRVFDVTERTALHRHLTGRTCHKCGTQLRDT
IVHFGERGTLGQPLNWEAATEAASKADTILCLGSSLKVLKKYPRLWCMTK
PPSRRPKLYIVNLQWTPKDDWAALKLHGKCDDVMRLLMDELGLEIPVYNR
WQDPIFSLATPLRAGEEGSHSRKSLCRSREEPPPGDQSAPLASATPILGG
WFGRGCAKRAKRKKAA
GenBank ® GI:7657575, Acc: NP_036370.2, sirtuin 1;
sirtuin (silent mating type information regulation
2, S. cerevisiae, homolog) 1; sirtuin type 1;
sir2-like 1; SIR2alpha [Homo sapiens]

(SEQ ID NO:15)

MADEAALALQPGGSPSAAGADREAASSPAGEPLRKRPRRDGPGLERSPGE
PGGAAPEREVPAAARGCPGAAAAALWREAEAEAAAAGGEQEAQATAAAGE
GDNGPGLQGPSREPPLADNLYDEDDDDEGEEEEEAAAAAIGYRDNLLFGD
EIITNGFHSCESDEEDRASHASSSDWTPRPRIGPYTFVQQHLMIGTDPRT
ILKDLLPETIPPPELDDMTLWQIVINILSEPPKRKKRKDINTIEDAVKLL
QECKKIIVLTGAGVSVSCGIPDFRSRDGIYARLAVDFPDLPDPQAMFDIE
YFRKDPRPFFKFAKEIYPGQFQPSLCHKFIALSDKEGKLLRNYTQNIDTL
EQVAGIQRIIQCHGSFATASCLICKYKVDCEAVRGDIFNQVVPRCPRCPA
DEPLAIMKPEIVFFGENLPEQFHRAMKYDKDEVDLLIVIGSSLKVRPVAL
IPSSIPHEVPQILINREPLPHLHFDVELLGDCDVIINELCHRLGGEYAKL
CCNPVKLSEITEKPPRTQKELAYLSELPPTPLHVSEDSSSPERTSPPDSS
VIVTLLDQAAKSNDDLDVSESKGCMEEKPQEVQTSRNVESIAEQMENPDL
KNVGSSTGEKNERTSVAGTVRKCWPNRVAKEQISRRLDGNQYLFLPPNRY
IFHGAEVYSDSEDDVLSSSSCGSNSDSGTCQSPSLEEPMEDESEIEEFYN
GLEDEPDVPERAGGAGFGTDGDDQEAINEAISVKQEVTDMNYPSNKS
GenBank ® GI:6320163, Acc: NP_010242.1|regulator
of silencing at HML, HMR, telomeres, and rDNA;
Sir2p [Sacoharomyces cerevisiae]

(SEQ ID NO:2)

MTIPHMKYAVSKTSENKVSNTVSPTQDKDAIRKQPDDIINNDEPSHKKIK
VAQPDSLRETNTTDPLGHTKAALGEVASMELKPTNDMDPLAVSAASVVSM
SNDVLKPETPKGPIIISKNPSNGIFYGPSFTKRESLNARMFLKYYGAHKF
LDTYLPEDLNSLYIYYLIKLLGFEVKDQALIGTINSIVHINSQERVQDLG
SAISVTNVEDPLAKKQTVRLIKDLQRAINKVLCTRLRLSNFFTIDHFIQK
LHTARKILVLTGAGVSTSLGIPDFRSSEGFYSKIKHLGLDDPQDVFNYNI
FMHDPSVFYNIANMVLPPEKIYSPLHSFIKMLQMKGKLLRNYTQNIDNLE
SYAGISTDKLVQCHGSFATATCVTCHWNLPGERIFNKIRNLELPLCPYCY
KKRREYFPEGYNNKVGVAASQGSMSERPPYILNSYGVLKPDITFFGEALP
NKFHKSIREDILECDLLICIGTSLKVAPVSEIVNMVPSHVPQVLINRDPV
KHAEFDLSLLGYCDDIAAMVAQKCGWTIPHKKWNDLKNKNFKCQEKDKGV
YVVTSDEHPKTL

A number of embodiments of the invention have been described. Nevertheless, it will be understood that various modifications may be made without departing from the spirit and scope of the invention. Accordingly, other embodiments are within the scope of the following claims.

What is claimed is:

1. A method for evaluating a test compound, the method comprising:
(a) providing a sample comprising:
(i) a nicotinamide-releasing activity,
(ii) a donor substrate (e.g., NAD), wherein the donor substrate comprises a nicotinamide moiety, and
(iii) a test compound;
(b) maintaining the sample under preselected conditions;
(c) contacting the sample to a matrix that preferentially interacts with the donor substrate relative to nicotinamide; and
(d) evaluating (i′) components of the contacted sample that do not interact with the matrix, or (ii′) components of the contacted sample that do interact with the matrix.

2. The method of claim 1 wherein the evaluating comprises assessing a parameter characteristic of components of the contacted sample that do not interact with the matrix.

3. The method of claim 1 wherein the parameter is a function of nicotinamide concentration.

4. The method of claim 1, where the contacting comprises separating components of the contact sample that interact with the matrix from the matrix, thereby separating the donor substrate from the sample

5. The method of claim 1, wherein the nicotinamide releasing enzyme comprises CD38, CD157, poly[ADP-ribose] polymerase (PARP), or an NAD glycohydrolase, or an enzymatically active fragment thereof.

6. The method of claim 1, wherein the nicotinamide releasing enzyme comprises a sirtuin or an enzymatically active fragment thereof.

7. The method of claim 1, wherein the matrix covalently bonds to the donor substrate.

8. The method of claim 1, wherein the matrix selectively interacts with compounds having 1,2-diols.

9. The method of claim 8, wherein the matrix includes a boronate group.

10. The method of claim 9, wherein the boronate group is:

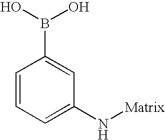

11. The method of claim 1, wherein the donor substrate is NAD, NADH, NADP, or NADPH.

12. The method of claim 1, wherein the nicotinamide-releasing activity is associated with a deacetylase activity.

13. The method of claim 1, wherein the nicotinamide-releasing activity is NAD hydrolase activity.

14. A method of evaluating nicotinamide in a sample, the method comprising:
(a) providing a sample;
(b) contacting the sample with a NAD-binding matrix, wherein the matrix does not also bind nicotinamide, under conditions that allow the NAD to bind the matrix; and
(c) detecting nicotinamide after the contacting.

15. A method for evaluating a test compound, the method comprising:
(a) providing a sample comprising: (i) a sample having nicotinamide-releasing activity; and (ii) a donor substrate, wherein the donor substrate comprises a nicotinamide moiety;
(b) maintaining the sample under preselected conditions;
(c) contacting the sample with a nicotinamide-modifying activity under conditions that allow the nicotinamide-modifying activity to react with the nicotinamide; and
(d) detecting a product of a reaction catalyzed by the nicotinamide-modifying activity or a nicotinamide that has been modified by the enzyme.

16. The method of claim 15, wherein the nicotinamide-modifying activity is nicotinamide deamidase activity.

17. The method of claim 16, wherein the product is ammonia.

18. The method of claim 17, wherein the detecting ammonia comprises:
contacting the reaction mixture with o-phthaldialdehyde (OPA); and
detecting optical density in the reaction mixture, thereby detecting levels of ammonia released.

19. The method of claim 15, wherein the nicotinamide-modifying enzyme is nicotinamide N-methyl transferase, and wherein the modified nicotinamide is detected.

20. The method of claim 19, wherein the contacting step further comprises the steps of: contacting the sample with acetophenone/KOH and formic acid, and heating the sample.

21. The method of claim 15, wherein the sample further comprises (iii) a test compound.

22. A method of detecting histone deacetylase activity, the method comprising:
providing a sample having histone deacetylase activity;
maintaining the sample under preselected conditions; and
detecting nicotinamide.

23. The method of claim 21, wherein the sample further comprises a donor substrate, wherein the donor substrate comprises a nicotinamide moiety and wherein the maintaining further comprises separating the nicotinamide from the donor substrate by binding the sample to a matrix that selectively interacts with the donor substrate but not the nicotinamide, wherein the binding is performed under conditions that allow the unreacted donor substrate to bind the matrix.

24. The method of claim 21, wherein the sample further comprises a donor substrate, wherein the donor substrate comprises a nicotinamide moiety; the maintaining further comprises contacting the sample with a nicotinamide-modifying enzyme under conditions that allow the nicotinamide-modifying enzyme to react with the nicotinamide; and the detecting nicotinamide comprises detecting one of: nicotinamide that has been modified by the enzyme, or a byproduct of the nicotinamide-modifying enzyme.

25. A method of evaluating a compound, the method comprising:
evaluating the effect of the compound on a parameter of a first animal cell that has a first level of expression or activity of a sirtuin; and
evaluating the effect of the compound on a parameter of a second animal cell that has a second level of expression or activity of a sirtuin.

26. The method of claim 25 further comprising: identifying the compound as a candidate compound if the compound has a differential effect on the second cell compared to the first cell.

27. The method of claim 25 wherein the sirtuin is Sirt1.

28. The method of claim 25 wherein the parameter is associated with a proliferative function.

29. The method of claim 25 wherein the first level of sirtuin expression or activity is greater than the second level of sirtuin expression or activity.

30. The method of claim 25 wherein the second level of sirtuin expression or activity is less than 50% of that of the first level of sirtuin expression or activity.

31. The method of claim 25 wherein the second level of sirtuin expression corresponds to no expression or no activity.

32. The method of claim 25 wherein the first animal cell is derived from a wild-type mouse and the second animal cell is derived from a sirtuin knockout mouse.
